(19) United States
(12) Patent Application Publication
(10) Pub. No.: US 2025/0380615 A1
(43) Pub. Date: Dec. 11, 2025

(54) IMPLANTABLE PIEZOELECTRIC ULTRASOUND STIMULATOR DEVICE AND RELATED SYSTEMS, STRUCTURES, AND METHODS

(71) Applicant: Massachusetts Institute of Technology, Cambridge, MA (US)

(72) Inventors: Canan Dagdeviren, Cambridge, MA (US); Jason Frank Hou, Cambridge, MA (US); Md Osman Goni Nayeem, Medford, MA (US)

(73) Assignee: Massachusetts Institute of Technology, Cambridge, MA (US)

(21) Appl. No.: 19/228,000

(22) Filed: Jun. 4, 2025

Related U.S. Application Data
(60) Provisional application No. 63/656,319, filed on Jun. 5, 2024.
(60) Provisional application No. 63/656,695, filed on Jun. 6, 2024.

(51) Int. Cl. H10N 30/88; A61N 7/00; A61N 7/02; H10N 30/02; H10N 30/063; H10N 30/088; H10N 30/853; H10N 39/00

(52) U.S. Cl. CPC H10N 30/883; A61N 7/02; H10N 30/02; H10N 30/063; H10N 30/088; H10N 30/8542; A61N 2007/0026; H10N 39/00

(57) ABSTRACT

Disclosed herein are example implantable piezoelectric ultrasound stimulator devices and related systems, structures, and methods. An implantable piezoelectric ultrasound stimulator device may comprise a piezoelectric film, a cavity, and electrodes that cause the piezoelectric film to generate ultrasound waves. The piezoelectric film, cavity, and electrodes may be encapsulated in a biocompatible polymer. Implantation of such a device in the brain and generation of ultrasound waves may stimulate neurons in the brain. Also disclosed herein are example structures for such a device. Further disclosed herein are example methods of making example implantable piezoelectric ultrasound stimulator devices disclosed herein. Still further disclosed herein are example systems for operating example implantable piezoelectric ultrasound stimulator devices disclosed herein.

### CROSS REFERENCE TO RELATED APPLICATIONS

This application claims the benefit of priority under 35 U.S.C. § 119 of U.S. Provisional Application No. 63/656,319, filed on Jun. 5, 2024, and U.S. Provisional Application No. 63/656,695, filed on Jun. 6, 2024. The entire contents of these applications are incorporated by reference herein.

### STATEMENT REGARDING FEDERALLY SPONSORED RESEARCH

N/A.

### BACKGROUND

Deep brain stimulation, by implanted electrodes that deliver electrical pulses to the brain, is often used to treat Parkinson's disease and other neurological disorders. The electrodes are often millimeters thick and are used to activate dopamine-producing cells in a brain region called the substantia nigra. However, once implanted in the brain, the electrodes eventually begin to corrode, and scar tissue builds up around the implanted electrodes. This scar tissue can interfere with the electrical impulses from the electrodes, and may require that the electrodes be removed.

### SUMMARY

Disclosed herein are example implantable piezoelectric ultrasound stimulator devices and related systems, structures, and methods. For example, an implantable piezoelectric ultrasound stimulator device may comprise a piezoelectric film, a cavity, and electrodes that cause the piezoelectric film to generate ultrasound waves. The piezoelectric film, cavity, and electrodes may be encapsulated in a biocompatible polymer. Implantation of such a device in the brain and generation of ultrasound waves may stimulate neurons in the brain. Also disclosed herein are example structures for such a device.

Further disclosed herein are example methods of making example implantable piezoelectric ultrasound stimulator devices disclosed herein. Still further disclosed herein are example systems for operating example implantable piezoelectric ultrasound stimulator devices disclosed herein.

As discussed herein, such a device uses ultrasounds to stimulate neurons in the brain, rather than electricity as in prior approaches. Example devices discussed herein can be implanted with a thin fiber, which may be easier to navigate to specific regions of the brain than in prior approaches and/or which may result in less tissue damage in the brain than in prior approaches. Example devices disclosed herein may also be less susceptible to corrosion and biofouling, as the electrode surfaces of example devices disclosed herein may not be exposed to the brain. Such a device may also be more power efficient than devices used in prior approaches. Moreover, as further discussed herein, example devices disclosed herein may be manufactured or controlled for use in specific regions of the brain.

In accordance with some embodiments, there is provided an implantable piezoelectric ultrasound stimulator device. The implantable piezoelectric ultrasound stimulator device comprises a first electrode and a second electrode. The implantable piezoelectric ultrasound stimulator device also comprises a piezoelectric film disposed between the first electrode and the second electrode. The implantable piezoelectric ultrasound stimulator device further comprises a biocompatible polymer that encapsulates the first electrode, the second electrode, the piezoelectric film, and a cavity.

In some embodiments, the biocompatible polymer comprises SU-8.

In further embodiments, the biocompatible polymer comprises a backing layer, a cavity layer forming the cavity, a membrane layer, and a top layer.

In still further embodiments, the piezoelectric film comprises a biocompatible ceramic.

In some embodiments, the piezoelectric film comprises potassium sodium niobate (KNN).

In further embodiments, the implantable piezoelectric ultrasound stimulator device comprises a piezoelectric micromachined ultrasound transducer (pMUT), the pMUT configured to generate and direct ultrasound waves in a direction away from an exposed planar surface of the top layer.

In still further embodiments, the implantable piezoelectric ultrasound stimulator device is configured to stimulate neurons in a brain.

In some embodiments, the implantable piezoelectric ultrasound stimulator device is less than 50 micrometers (μm) thick and less than 200 μm wide.

In further embodiments, the cavity is positioned on one side of the piezoelectric film and is filled with air.

In still further embodiments, the first electrode, the second electrode, the cavity, and the piezoelectric film together form one ultrasound element of an array of ultrasound elements in the implantable piezoelectric ultrasound stimulator device, each of the ultrasound elements in the implantable piezoelectric ultrasound stimulator device comprising at least two electrodes, a cavity, and a piezoelectric film.

Furthermore, in accordance with embodiments of the present disclosure, there is provided a system. The system comprises the implantable piezoelectric ultrasound stimulator device, a controller, and a power source.

In some embodiments, the controller is configured to deliver a voltage from the power source to the first electrode or the second electrode.

In further embodiments, the voltage is one of a sinusoidal voltage or a pulsed voltage.

In still further embodiments, the controller is further configured to control a frequency at which the sinusoidal voltage or pulsed voltage is delivered.

In some embodiments, the first electrode, the second electrode, the cavity, and the piezoelectric film together form one ultrasound element of an array of ultrasound elements sin the implantable piezoelectric ultrasound stimulator device, each of the ultrasound elements in the implantable piezoelectric ultrasound stimulator device comprising at least two electrodes, a cavity, and a piezoelectric film, and wherein the controller is configured to individually control the ultrasound elements in the array to form an ultrasound beam focused in a specific direction.

In further embodiments, the controller and the power source are implantable, and the controller is configured to communicate wirelessly with a control device.

In still further embodiments, the controller is configured to control the implantable piezoelectric ultrasound stimulator device over a cable.

Still further, in accordance with some embodiments of the present disclosure, there is provided a method of making an implantable piezoelectric ultrasound stimulator device. The method comprises providing a stack of layers on top of a first substrate, the stack of layers comprising at least a first electrode layer in contact with the top of the first substrate and comprising at least one first electrode, a second electrode layer comprising at least one second electrode, and a piezoelectric layer between the first electrode layer and the second electrode layer and comprising at least one piezoelectric film. The method also comprises coating the stack of layers and a portion of the first substrate with an anchor material. The method further comprises undercutting the stack of layers by removing at least a portion of the first substrate. The method still further comprises removing the stack of layers and at least a portion of the anchor material from the first substrate. The method also comprises pressing the stack of layers and the at least a portion of the anchor material onto a first layer of a biocompatible polymer, the first layer of the biocompatible polymer positioned atop a release layer and a second substrate. The method further comprises removing the at least a portion of the anchor material from the stack of layers. The method still further comprises coating a second layer of the biocompatible polymer onto the stack of layers and the first layer of the biocompatible polymer, leaving openings to the at least one first electrode and the at least one second electrode. The method also comprises depositing and etching metal interconnects and bond pads for connecting the at least one first electrode and the at least one second electrode to external circuitry. The method further comprises coating a third layer of the biocompatible polymer onto the metal interconnects and the second layer of the biocompatible polymer. The method still further comprises removing the second substrate from the release layer, and removing the release layer.

In some embodiments, the method further comprises patterning a cavity on top of the third layer of the biocompatible polymer. The method also comprises coating a fourth layer of the biocompatible polymer onto the third layer of the biocompatible polymer, except where the cavity was patterned. The method further comprises bonding the fourth layer of the biocompatible polymer to a fifth layer of the biocompatible polymer on a third substrate. The method still further comprises removing the third substrate.

In further embodiments, the biocompatible polymer comprises SU-8 and the at least one piezoelectric film comprises potassium sodium niobate (KNN).

It should be appreciated that individual elements of different embodiments described herein may be combined to form other embodiments not specifically set forth above. Various elements, which are described in the context of a single embodiment, may also be provided separately or in any suitable sub-combination. It should also be appreciated that other embodiments not specifically described herein are also within the scope of the following claims.

### BRIEF DESCRIPTION OF THE DRAWINGS

The manner of making and using the disclosed subject matter may be appreciated by reference to the detailed description in connection with the drawings, in which like reference numerals identify like elements.

FIG. 1A is a perspective view showing elements of an example implantable piezoelectric ultrasound stimulator device, consistent with embodiments of the disclosure herein.

FIG. 1B is a block diagram of a cross-sectional view of an example implantable piezoelectric ultrasound stimulator device, consistent with embodiments of the disclosure herein.

FIG. 2A is a perspective view of an example implantable piezoelectric ultrasound stimulator device coupled to a controller.

FIG. 2B is an enlarged perspective view of a portion of the implantable piezoelectric ultrasound stimulator device of FIG. 2A.

FIG. 2C is an enlarged perspective view of a portion of the implantable piezoelectric ultrasound stimulator device of FIG. 2A.

FIG. 2D is an illustration showing an example use of an example implantable piezoelectric ultrasound stimulator device in stimulating neurons in a mouse.

FIG. 3A is a graph of impedance and phase angle vs. frequency of an example implantable piezoelectric ultrasound stimulator device, showing the resonance frequency in two different mediums.

FIG. 3B is a graph of displacement vs. frequency of an example implantable piezoelectric ultrasound stimulator device in two different mediums when the applied voltage is a periodic chirp and when the applied voltage is a sinusoidal signal.

FIG. 4A is a graph of resonant frequency vs. cavity diameter for an example implantable piezoelectric ultrasound stimulator device provided in accordance with the concepts described herein.

FIG. 4B is a graph of displacement vs. voltage for an example implantable piezoelectric ultrasound stimulator device provided in accordance with the concepts described herein.

FIG. 4C is a graph of displacement vs. voltage for an example implantable piezoelectric ultrasound stimulator devices provided in accordance with the concepts described herein having different cavity sizes.

FIG. 5A is a plot of pressure as a function of height (Z) and radial distance (r) from an example implantable piezoelectric ultrasound stimulator device provided in accordance with the concepts described herein.

FIG. 5B is a graph of pressure vs. distance from transducer, comparing simulated and experimentally measured pressures at different distances from an example implantable piezoelectric ultrasound stimulator device provided in accordance with the concepts described herein.

FIG. 6 is a graph of temperature change in a water medium vs. time for an operational implantable piezoelectric ultrasound stimulator device provided in accordance with the concepts described herein.

FIG. 7A is a graph of applied voltages vs. time illustrating the amount of applied voltage require to maintain a steady displacement of an example implantable piezoelectric ultrasound stimulator device provided in accordance with the concepts described herein.

FIG. 7B is a graph of normalized displacement vs. time for an example implantable piezoelectric ultrasound stimulator device provided in accordance with the concepts described herein when the applied voltages of FIG. 7A were used.

FIG. 8A is a graph showing the normalized displacement of an example implantable piezoelectric ultrasound stimulator device over days of a fatigue test.

FIG. 8B is a graph showing the applied voltage used to maintain a steady displacement of an example implantable piezoelectric ultrasound stimulator device over days of a fatigue test.

FIG. 8C is a graph showing the normalized displacement of an example implantable piezoelectric ultrasound stimulator device over days of a fatigue test when the voltages of FIG. 8B were used.

FIG. 9A is a diagram of a two-photon imaging setup with a coronal hippocampal slice and an example implantable piezoelectric ultrasound stimulator device under magnification, where artificial cerebrospinal fluid (aCSF) is circulated throughout the bath and a function generator connected to the leads outside the bath provide excitation for ultrasonic stimulation.

FIG. 9B is a graph of fluorescence change in three different regions of interest over time using the setup of FIG. 9A.

FIG. 10 is a graph showing the results of an experiment measuring cFos+protein in the dorsal CA1 (dCA1) layer of the hippocampal formation after stimulation with an example implantable piezoelectric ultrasound stimulator device operating at a frequency of 500 kilohertz (kHz), after stimulation with an example implantable piezoelectric ultrasound stimulator device operating at a frequency of 500 kHz with a 10% duty factor, and without stimulation.

FIG. 11A is a graph showing the results of an experiment recording Z-score DA2m fluorescence in a control region of a brain where stimulation from an example implantable piezoelectric ultrasound stimulator device is turned on and off.

FIG. 11B is a graph showing the results of an experiment recording Z-score DA2m fluorescence in the substantia nigra pars compacta (SNc) region of a brain where stimulation from an example implantable piezoelectric ultrasound stimulator device is turned on and off.

FIG. 11C is a graph showing the area under the curve of FIGS. 10A and 10B prior to stimulation and during stimulation using the example implantable piezoelectric ultrasound stimulator device.

FIG. 12A is a diagram of a portion of a process for making an example implantable piezoelectric ultrasound stimulator device.

FIG. 12B is a diagram of another portion of the process for making an example implantable piezoelectric ultrasound stimulator device.

FIG. 13 is a view of an example implantable piezoelectric ultrasound stimulator device comprising an array of piezoelectric elements.

FIG. 14 is a block diagram of an example system for controlling an implantable piezoelectric ultrasound stimulator device provided in accordance with the concepts described herein.

FIG. 15 is a block diagram of an example implantable piezoelectric stimulator device provided in accordance with the concepts described herein.

The drawings are not necessarily to scale, or inclusive of all elements of a system, emphasis instead generally being placed upon illustrating the concepts, structures, and techniques sought to be protected herein.

### DETAILED DESCRIPTION

For convenience, certain introductory concepts and terms used in the specification are collected here.

As used herein, the term “biocompatible” refers to substances that are substantially nontoxic to cells in the quantities and at the location used and/or that do not elicit or cause a significant deleterious or untoward effect on the recipient's body at the location used (e.g., an unacceptable immunological or inflammatory reaction, unacceptable scar tissue formation). In some embodiments, a substance may be considered to be “biocompatible” if its placement near cells in vitro or in vivo results in less than or equal to about 20% cell death relative to a baseline control where the substance is not placed near cells in vitro or in vivo. For example, SU-8, discussed herein, is a commonly used epoxy-based negative photoresist material, and is generally considered to be a “biocompatible” material. See, e.g., “In Vitro and In Vivo Evaluation of SU-8 Biocompatibility,” Nemani et al., Mater. Sci. Eng. C. Mater. Biol. Appl., October 2013, which is incorporated herein by reference. As another example, potassium sodium niobate (KNN), discussed herein, is generally considered to be a “biocompatible” material. See, e.g., “Fabrication of Biocompatible Potassium Sodium Niobate Piezoelectric Ceramic as an Electroactive Implant,” Chen et al., Materials, 2017, which is incorporated herein by reference.

As used herein, the term “ultrasound” refers to sound with frequencies greater than or equal to about 20 kilohertz (kHz).

As used herein, the term “piezoelectric” refers to an electric charge that accumulates in a solid material in response to an applied mechanical stress, or a mechanical change that occurs in a solid material in response to an applied electric field.

As used herein, the term “piezoelectric micromachined ultrasonic transducer” or “pMUT” refers to a type of transducer that uses a thin piezoelectric membrane to generate ultrasound waves, and that is fabricated using micromachining techniques, allowing for their miniaturization and integration into various systems.

As used herein, the term “micromachining” or “microfabrication” refers to techniques used to fabricate extremely small, typically microscopic (e.g., on the micrometer scale) structures.

As used herein, the term “implantable” refers to the capability of being implanted or designed to be implanted in a living body. In some embodiments described herein, an “implantable” device may be fully implanted within a living body. In other embodiments described herein, an “implantable” device may be a device in which a portion of the device is implanted within a living body, while another portion of the device is external to the living body.

Example devices are discussed herein as being capable of being implanted and of generating ultrasound waves in the brains of certain living bodies, such as in a living human brain or in a living brain of a test animal. However, the disclosure is not so limited. Example implantable devices discussed herein may be used in brains of a wide variety of different living organisms, or for study in ex vivo brain tissue slices.

Precise and reversible spatiotemporal control of neural activity is an ultimate goal of many neurostimulation strategies, both in therapeutic applications and in neuroscience research. Current neurostimulation strategies may be broadly divided into two categories: (i) non-invasive strategies and (ii) invasive strategies. Some existing non-invasive strategies used in clinical treatment include transcranial magnetic stimulation (TMS), transcranial current stimulation (TCS), and transcranial-focused ultrasound (tFUS). While these strategies may avoid surgery and associated recurrent risks, current TMS and TCS approaches may be problematic, in that electromagnetic energy generated by these strategies may scatter through bone and tissue attenuation. Unobstructed transcranial focused ultrasound (tFUS) strategies may be capable of achieving millimeter-scale resolution in neural tissue and may penetrate several centimeters to excite neurons by affecting mechanoreceptive ion channels and other membrane-bound ion channels. Furthermore, the ability to quickly evaluate potential stimulation targets and to adjust various ultrasound parameters, such as frequency and acoustic intensity, may make tFUS an advantageous approach for neurostimulation therapy in patients with conditions such as Alzheimer's disease, epilepsy, or depression. For example, to achieve a balance between skull transmission and spatial selectivity, most significant modulations of neurons with ultrasound have been reported at frequencies less than 1 megahertz (MHz), and particularly with 500 kilohertz (kHz) with pressures at or above 100 kilopascal (kPa). However, ultrasound, when transmitted from outside a skull (e.g., human skull), may face significant scattering and reflection from the skull's high acoustic impedance, which may cause off-target stimulation via conduction through bone and auditory pathways, and which may even cause traumatic, irreversible brain injury.

Invasive strategies using implantable devices that allow electrical and/or chemical modulation of the brain may lead to significant advancements in treating neurological and psychiatric disorders. For example, electrical deep brain stimulation (DBS) may induce reversible activation of neurons. DBS, by implanted electrodes that deliver electrical pulses to the brain, is often used to treat Parkinson's disease and other neurological disorders. The electrodes are often millimeters thick and are used to activate dopamine-producing cells in a brain region called the substantia nigra. However, DBS may be limited by anisotropic charge transfer across the brain's ionic medium to regions proportional to the size of the electrode used in the implantable device. Moreover, both the charge provided by the electrodes of such a device and the sensitivity of the surrounding tissue may decrease significantly over time due to biofouling and corrosion, which may limit the longevity of the device. That is, once implanted in the brain, the electrodes of such a device may eventually begin to corrode, and scar tissue may build up around the implanted electrodes. This scar tissue may interfere with the electrical impulses from the electrodes, and may require that the electrodes be removed.

Invasive strategies based on optogenetics may provide minimally-invasive neurostimulation with high spatiotemporal resolution and cell-type specificity. Such strategies may seek to introduce and stimulate opsins, or light-sensitive receptors, in a living body. However, the potential of these strategies for clinical translation may be limited. First, the long-term safety and efficacy of opsin expression in the primate nervous system remains poorly characterized. Second, the transgenic delivery of opsins may require local or systemic infections, which may pose a risk of immunogenicity. Additionally, optical fibers used to stimulate opsins with various frequencies of light may produce light scattering that is difficult to minimize, posing a risk of off-target neural activation or inhibition.

Reports of miniaturized ultrasonic neurostimulation devices have shown that directed ultrasound energy can activate cultured neurons and neurons in brain slices. However, the platforms discussed in these reports are not optimized for implantation in the deep brain, due to their rigid form factors, material composition, or high power requirements. Outside of implanted electrical stimulation, there remains a lack of non-genetic techniques for anatomically localized modulation of brain regions, such as deep subcortical brain regions.

Development of a technique for safe, widespread non-immunogenic delivery in the brain remains a challenge for clinical translation. A robust, miniaturized, scalable implant system that has the capability to non-genetically and locally modulate neurons in brain regions is needed to address the deficiencies of current approaches and to reach high standards of safety and longevity.

Disclosed herein are example implantable piezoelectric ultrasound stimulator devices (also sometimes referred to herein as “ImPULS” devices) and related systems, structures, and methods. For example, an implantable piezoelectric ultrasound stimulator device may comprise a piezoelectric film, a cavity, and electrodes that cause the piezoelectric film to generate ultrasound waves. The piezoelectric film, cavity, and electrodes may be encapsulated in a biocompatible polymer. Implantation of such a device in the brain and generation of ultrasound waves may stimulate neurons in the brain. Also disclosed herein are example structures for such a device.

Further disclosed herein are example methods of making example implantable piezoelectric ultrasound stimulator devices disclosed herein. Still further disclosed herein are example systems for operating example implantable piezoelectric ultrasound stimulator devices disclosed herein.

The example implantable piezoelectric ultrasound stimulator devices and related systems, structures, and methods disclosed herein may address problems associated with current and prior approaches to neurostimulation.

For example, implantable piezoelectric ultrasound stimulator devices disclosed herein may use ultrasounds to stimulate neurons in the brain, rather than electricity as in some prior approaches. Example devices discussed herein can be implanted with a thin fiber, which may be easier to navigate to specific regions of the brain than in prior approaches and/or which may result in less tissue damage in the brain than in prior approaches. Example devices disclosed herein may also be less susceptible to corrosion and biofouling, as the electrode surfaces of the example devices disclosed herein may not be exposed to the brain. Example devices disclosed herein may also be more power efficient than devices used in prior approaches. Moreover, as further discussed herein, example devices disclosed herein may be customized (e.g., manufactured or controlled) for use in specific regions of the brain.

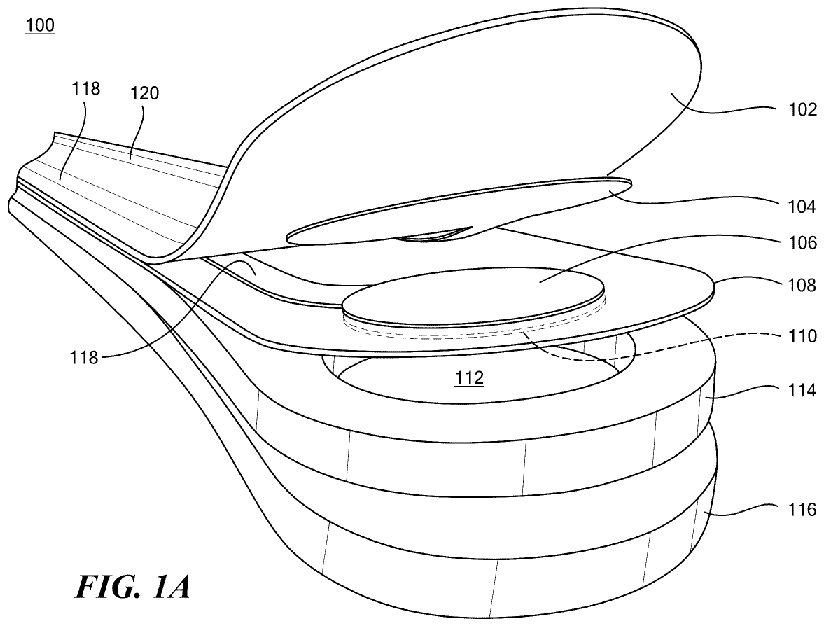

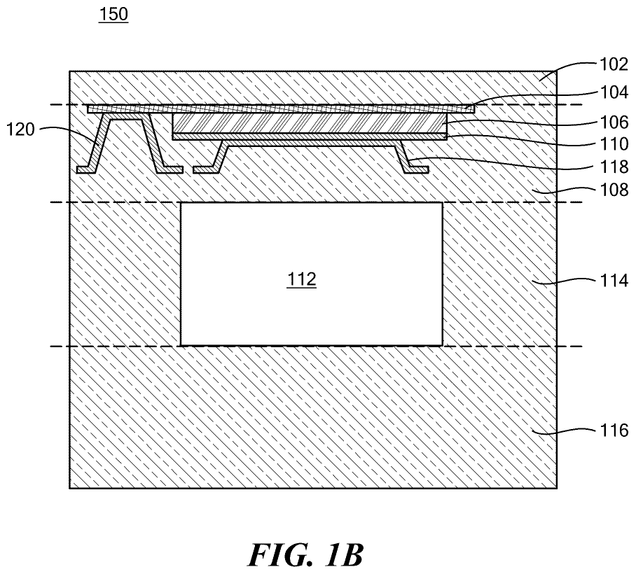

FIG. 1A is a diagram showing elements of an example implantable piezoelectric ultrasound stimulator device 100 provided in accordance with the concepts sought to be protected herein and consistent with embodiments of the disclosure herein. The diagram of FIG. 1A shows a peeled view of the example implantable piezoelectric ultrasound stimulator device, to show the different layers of the device.

As shown in FIG. 1A, example implantable piezoelectric ultrasound stimulator device 100 may comprise one or more backing layers (e.g., backing layer 116), one or more cavity layers (e.g., cavity layer 114), one or more cavities (e.g., cavity 112), one or more bottom electrodes (e.g., bottom electrode 110 (not shown in FIG. 1A)), one or more membrane layers (e.g., membrane layer 108), one or more piezoelectric films (e.g., piezoelectric film 106), one or more top electrodes (e.g., top electrode 104), one or more top films (e.g., top film 102), and conductive traces (e.g., conductive traces 118, 120).

In some embodiments, the one or more backing layers, one or more cavity layers, one or more membrane layers, and one or more top layers may be formed of a biocompatible material, such as a biocompatible polymer. In some embodiments, each of these layers may be formed of the same type of biocompatible material. In other embodiments, different ones of these layers may be formed of different types of biocompatible material. In some embodiments, each of these layers may be formed of SU-8, which is a type of high-contrast, epoxy-based negative photoresist suitable for use in micromachining and microelectronics applications and which is generally considered to be a biocompatible material. However, the disclosure is not so limited. For example, in some embodiments each of the layers may be formed of polyimide or another type of biocompatible material.

In some embodiments, the one or more cavities (e.g., cavity 112) may be filled with air, though the disclosure is not so limited. For example, the one or more cavities may alternatively be filled or coated with a dense metal, such as platinum, gold, tungsten, lead, or any other type of dense metal. In some embodiments, the one or more cavities may be filled or coated with SiO₂. In some embodiments, the one or more cavities may be filled or coated with a particle-filled epoxy that comprises air, one or more of the aforementioned dense metals, and/or SiO₂. Moreover, although a single cavity 112 is illustrated in FIG. 1A, the disclosure is not so limited. In some embodiments, a plurality of cavities may be included in an implantable piezoelectric ultrasound stimulator device, such as an array of cavities, which may all be filled with the same material (e.g., air) or which may be filled with different materials. Additionally, although cavity 112 is illustrated in FIG. 1A as being circular in shape, the disclosure is not so limited. A cavity 112 may have any shape or dimension, depending on the application and the results sought to be achieved with the cavity.

The one or more piezoelectric films (e.g., piezoelectric film 106) may be formed of a piezoelectric material. In some embodiments, the one or more piezoelectric films may be formed of potassium sodium niobate ((K,Na)NbO₃) or (KNN), which is a piezoelectric ceramic material that is generally considered to be biocompatible and that has good piezoelectric properties (e.g., good d₃₃, e₃₁, d₃₁, durability, and Curie temperature properties). For example, KNN is lead-free, has high piezoelectric coefficients (e₃₁, d₃₁) and durability (direct current (DC) stress lifetime of greater than 24 hours at 200° C. and 30 kV/cm), and has a Curie temperature of 350° C. Thus, KNN has properties that may exceed those of other commercially available doped lead zirconate titanate (PZT) and polymer-based piezoelectric materials. Moreover, KNN has proven biocompatibility and non-toxicity and is commercially available. However, the disclosure is not limited to using KNN as the one or more piezoelectric films. The one or more piezoelectric films may instead be formed of, for example, PZT, barium titanate (BaTiO₃), zinc oxide (ZnO), doped zinc oxide, aluminum nitride (AlN), trifluoroethylene (PVDF-TrFE), aluminum scandium nitride (AlScN), or any other known piezoelectric material. In some embodiments, a plurality of piezoelectric films may be included in an implantable piezoelectric ultrasound stimulator device, such as an array of piezoelectric films, which may all be formed of the same material (e.g., KNN) or which may be formed of different materials. Additionally, although piezoelectric film 106 is illustrated in FIG. 1A as being circular in shape, the disclosure is not so limited. A piezoelectric film 106 may have any shape or dimension, depending on the application and the results sought to be achieved with the piezoelectric film.

The one or more bottom electrodes (e.g., bottom electrode 110) and the one or more top electrodes (e.g., top electrode 104) may be formed of one or more electrically conductive materials, such as gold (Au), chromium (Cr), chromium/gold (Cr/Au), platinum (Pt), silver (Ag), copper (Cu), titanium (Ti), brass, a thin film stack of titanium/platinum/gold (Ti/Pt/Au), aluminum (Al), carbon (C), graphite, a conductive polymer, a conductive metal alloy, a conductive graphene-based material, or a conductive hybrid nanomaterial. In some embodiments, the one or more bottom electrodes and the one or more top electrodes may be made of different materials. For example, the one or more bottom electrodes may comprise chromium/gold (Cr/Au), while the one or more top electrodes may comprise platinum (Pt). In some embodiments, a plurality of bottom electrodes may be included in an implantable piezoelectric ultrasound stimulator device, such as an array of bottom electrodes, which may all be formed of the same material (e.g., chromium/gold) or which may be formed of different materials. In some embodiments, a plurality of top electrodes may be included in an implantable piezoelectric ultrasound stimulator device, such as an array of top electrodes, which may all be formed of the same material (e.g., platinum) or which may be formed of different materials.

Although FIG. 1A shows a peeled view of example implantable piezoelectric ultrasound stimulator device 100 to show the different layers of the device, the assembled device would not be structured in this peeled fashion. Rather, the one or more backing layers (e.g., backing layer 116), one or more cavity layers (e.g., cavity layer 114), one or more membrane layers (e.g., membrane layer 108) and one or more top layers (e.g., top layer 102) would be bonded together, so as to encapsulate (i.e., completely surround) the one or more cavities (e.g., cavity 112), one or more bottom electrodes (e.g., bottom electrode 110), one or more piezoelectric films (e.g., piezoelectric film 106), one or more top electrodes (e.g., top electrode 104), and conductive traces (e.g., conductive traces 118, 120). Fully encapsulating these elements in these layers, which as discussed above may be made of a biocompatible material (e.g., SU-8), may provide for an implantable piezoelectric ultrasound stimulator device where only biocompatible materials are exposed to tissues of the brain when the device is implanted. In some embodiments, the one or more backing layers, one or more cavity layers, one or more membrane layers, and/or one or more top layers may be bonded together using heat bonding, causing the layers to fuse together. In other embodiments, the one or more backing layers, one or more cavity layers, one or more membrane layers, and/or one or more top layers may be bonded together in another fashion, such as through use of an adhesive.

A piezoelectric film (e.g., piezoelectric film 106) may be positioned between a bottom electrode (e.g., bottom electrode 110) and a top electrode (e.g., top electrode 104). The bottom electrode may be coupled to a conductive trace (e.g., conductive trace 118) and the top electrode may be coupled to another conductive trace (e.g., conductive trace 120). A power source may be coupled to the conductive traces to apply voltages to the conductive traces. Application of these voltages may cause the top and bottom electrodes together to act as a capacitor, with a voltage applied across the piezoelectric film. Changes in these voltages may cause the piezoelectric film to deform. By applying these voltage changes at a particular frequency, the implantable piezoelectric ultrasound stimulator device may operate as a piezoelectric micromachined ultrasonic transducer (pMUT), generating ultrasound waves. When implanted, the cavity (e.g., cavity 112) may act as acoustic reflector, and the cavity, along with the thickness of the backing layer (e.g., backing layer 116), may cause ultrasound waves to be generated and transmitted primarily in a direction away from the top layer (e.g., top layer 102) of the implantable piezoelectric ultrasound stimulator device (i.e., toward the top of the page of FIG. 1A). As will be further discussed herein, the frequency and acoustic amplitude of the ultrasound signals generated and transmitted by the implantable piezoelectric ultrasound stimulator device may be customized by varying the dimensions of the piezoelectric film, the dimensions of the cavity, or by varying the voltages and/or frequency of voltages applied to the electrodes of the implantable piezoelectric ultrasound stimulator device, as just some examples.

FIG. 1B is a diagram showing a cross-section 150 of an example implantable piezoelectric ultrasound stimulator device, consistent with embodiments of the disclosure herein. For example, cross-section 150 may be a cross-section of implantable piezoelectric ultrasound stimulator device 100 of FIG. 1A taken through top layer 102, top electrode 104, piezoelectric film 106, membrane layer 108, bottom electrode 110, cavity 112 and cavity layer 114, backing layer 116, and metal interconnects of traces 118, 120.

As discussed with respect to FIG. 1A, and as shown in FIG. 1B, the implantable piezoelectric ultrasound stimulator device may include one or more backing layers 116, one or more cavity layers 114, one or more membrane layers 108, and one or more top layers 102. As previously discussed, these layers may be formed of one or more biocompatible materials, such as biocompatible polymers (e.g., SU-8). Encapsulated within these layers may be one or more cavities 112 filled with a material (e.g., air) and disposed within the one or more cavity layers 114 between the one or more backing layers 116 and the one or more membrane layers 108. Also encapsulated within these layers may be one or more bottom electrodes 110, one or more top electrodes 104, traces 118, 120, and one or more piezoelectric films 106 disposed between (e.g., sandwiched between) one or more bottom electrodes 110 and one or more top electrodes 104. The one or more top electrodes may be formed of a conductive material, as discussed above with respect to FIG. 1A. The one or more bottom electrodes may be formed of a conductive material, as discussed above with respect to FIG. 1A. The one or more piezoelectric films may be formed of a piezoelectric material, such as biocompatible KNN, as discussed above with respect to FIG. 1A.

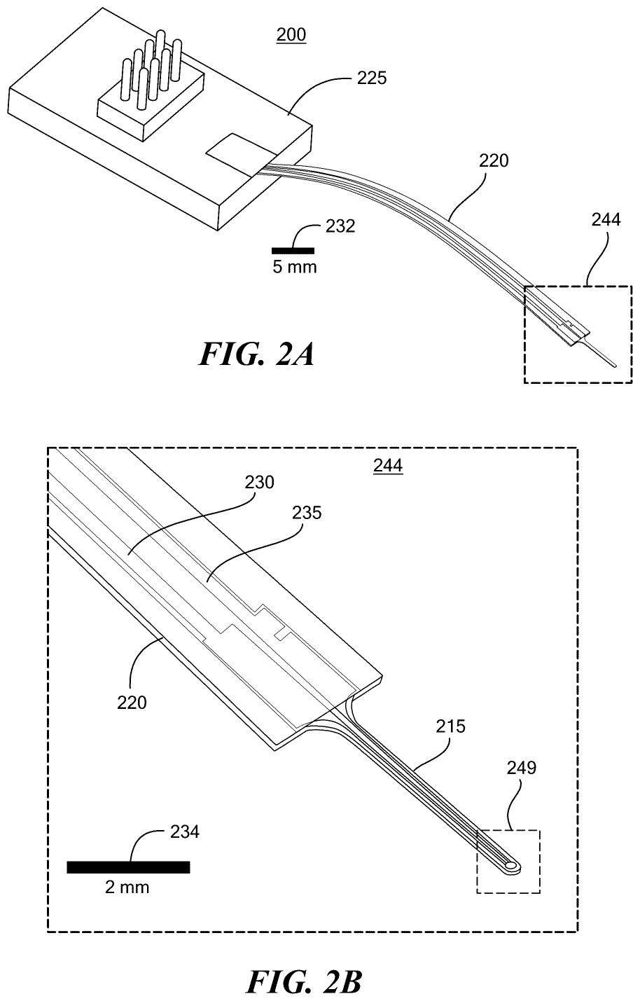

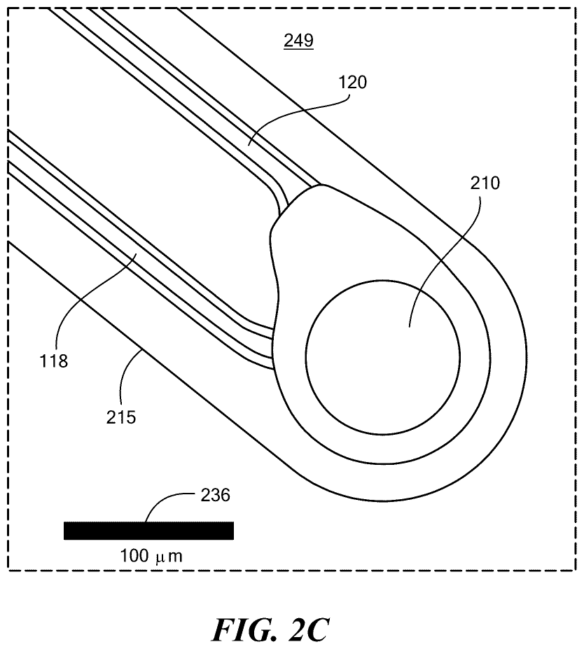

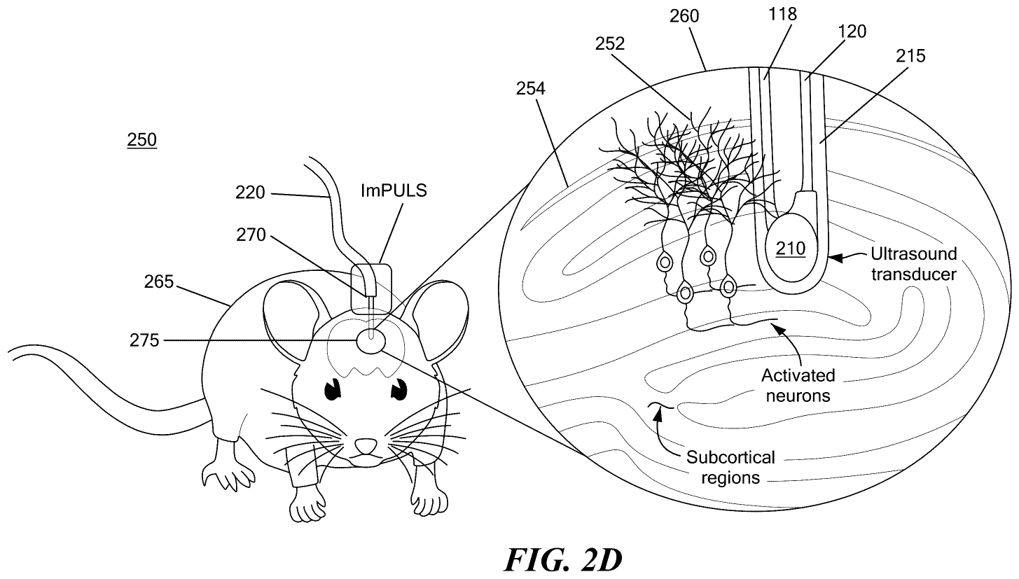

FIG. 2A is an illustration 200 of an example implantable piezoelectric ultrasound stimulator device (see 215 in FIGS. 2B, 2C) and a system for controlling implantable piezoelectric ultrasound stimulator device 215. Example implantable piezoelectric ultrasound stimulator device 215 may be constructed as shown and described with respect to FIGS. 1A and 1B. More specifically, implantable piezoelectric ultrasound stimulator device 215 may include a backing layer (e.g., backing layer 116), a cavity layer (e.g., cavity layer 114), a membrane layer (e.g., membrane layer 108), and a top layer (e.g., top layer 102), which may be comprised of SU-8. The backing layer, cavity layer, membrane layer, and top layer may be bonded together to encapsulate a cavity (e.g., cavity 112) that may be filled with air, a bottom electrode (e.g., bottom electrode 110) that may comprise chromium/gold, a top electrode (e.g., top electrode 104) that may comprise platinum, and a piezoelectric film (e.g., piezoelectric film 106) positioned between the bottom electrode and top electrode that may comprise KNN. As shown in FIGS. 2A-2C, conductive traces (e.g., conductive traces 118, 120) of element 215 may be coupled to a ribbon cable 220 comprising two conductive traces 230, 235, which may electrically connect the conductive traces of element 215 to a controller (e.g., printed board (PCB)) 225. Controller 225 may be configured to supply power (e.g., voltages) to the top and bottom electrode over the conductive traces.

Element 210 (see FIG. 2C) is the piezoelectric element (e.g., pMUT) of implantable piezoelectric ultrasound stimulator device 215. That is, element 210 comprises the cavity (e.g., cavity 112), bottom electrode (e.g., bottom electrode 110), top electrode (e.g., top electrode 104), and piezoelectric film (e.g., piezoelectric film 106) between the bottom electrode and top electrode.

Box 244 of FIG. 2A outlines implantable piezoelectric ultrasound stimulator device 215 and a section of ribbon cable 220, which is enlarged in FIG. 2B.

FIG. 2B is an enlarged perspective view of a portion of the implantable piezoelectric ultrasound stimulator device and portion of ribbon cable outlined with box 244 of FIG. 2A. As shown in FIG. 2B, ribbon cable 220 may comprise conductive traces 230, 235, and may be coupled to implantable piezoelectric ultrasound stimulator device 215.

Box 249 of FIG. 2B outlines a section of implantable piezoelectric ultrasound stimulator device 215 that comprises piezoelectric element 210 (the pMUT) (see FIG. 2C), which is enlarged in in FIG. 2C.

FIG. 2C is an enlarged perspective view of a portion of the implantable piezoelectric ultrasound stimulator device outlined with box 249 of FIG. 2B. As shown in FIG. 2C, implantable piezoelectric ultrasound stimulator device 215 may comprise piezoelectric element 210 (the pMUT) and conductive traces 118, 120. As also shown in FIG. 2C, piezoelectric element 210 and conductive traces 118, 120 may be encapsulated in a polymer (e.g., biocompatible polymer, such as SU-8) in implantable piezoelectric ultrasound stimulator device 215.

For reference, scale bar 232 of FIG. 2A may correspond to 5 millimeters (mm), scale bar 234 of FIG. 2B may correspond to 2 mm, and scale bar 236 of FIG. 2C may correspond to 100 μm.

As discussed above, implantable piezoelectric ultrasound stimulator device 215 may be encapsulated in a flexible biocompatible polymer, such as SU-8. Implantable piezoelectric ultrasound stimulator device 215 may be about as thick as a human hair. The thinness and flexibility of implantable piezoelectric ultrasound stimulator device 215 may make it easy to surgically navigate implantable piezoelectric ultrasound stimulator device 215 into the brain, to deliver piezoelectric element 210 to a desired region of the brain. Ribbon 220 and controller 225 may remain external to the brain.

Example implantable piezoelectric ultrasound stimulator device 215 of FIG. 2A was formed to include a single piezoelectric element (i.e., one cavity, one bottom electrode, one top electrode, and one piezoelectric film positioned between the bottom electrode and the top electrode). An example implantable piezoelectric ultrasound stimulator device 215 was formed to be 30 micrometers (μm) thick and 140 μm wide. The backing layer (e.g., backing layer 116) was formed to be 15 μm thick, the cavity layer (e.g., cavity layer 114) was formed to be 15 μm thick, the membrane layer (e.g., membrane layer 108) was formed to be 0.8 μm thick, and the top layer (e.g., top layer 102) was formed to be 0.5 μm thick. The cavity (e.g., cavity 112) was filled with air. The piezoelectric film (e.g., piezoelectric film 106) was formed of KNN and was formed to be 1 μm thick and 100 μm in diameter. The top electrode (e.g., top electrode 104) was formed of platinum that was 100 nanometers (nm) thick. The bottom electrode (e.g., bottom electrode 110) was formed of chromium/gold, with the chromium 10 nm thick and the gold 250 nm thick. Conductive traces 118, 120 were formed of chromium/gold, with the chromium 10 nm thick and the gold 250 nm thick. Of course, the disclosure is not limited to these dimensions or materials. A variety of different dimensions and/or materials for the different components of implantable piezoelectric ultrasound stimulator device 215 may be used, and may be varied depending on a desired result for a particular application. In some embodiments, an implantable piezoelectric ultrasound stimulator device may be formed to be less than 50 μm thick and less than 200 μm wide.

In order to maximize the vibration amplitude of the active membrane, the dimensions of the element geometries of implantable piezoelectric ultrasound stimulator device 215 of FIGS. 2A-2C were designed to replicate a pMUT with a pinned boundary device structure. Compared to bulk piezoceramics and silicon-based pMUTs, implantable piezoelectric ultrasound stimulator device 215 may have a thinner profile and lower Young's Modulus, which may better couple to soft brain tissue.

Without a pMUT, a simple thickness-mode resonance-dependence may make piezoceramic devices at the 500 kHz range millimeters thick and unfavorable for minimally-invasive neurostimulation. The single-element pMUT of implantable piezoelectric ultrasound stimulator device 215 was chosen to target adjacent neuron somas in the 50 μm hemispherical radius of the transducer for study. However, as will be further discussed herein, an implantable piezoelectric ultrasound stimulator device may be scaled in fabrication to produce flexible arrays for targeting larger regions of the brain tissue.

Implantable piezoelectric ultrasound stimulator device 215 of FIGS. 2A-2C was tested in the system of FIG. 2A as generating ultrasounds at a pressure of 59.2 kPa at 15 μm away from the device, and 100 kPa adjacent to the transducer, for the single piezoelectric element. The device was tested and remained functional after seven days in an accelerated (75° C.) phosphate-buffered saline (PBS) solution without incurring significant electrical and mechanical degradation. The device was also tested and determined to not cause temperature rise above safe tissue thresholds during ultrasound generation.

FIG. 2D is an illustration 250 showing an example use of an example implantable piezoelectric ultrasound stimulator device 215 in stimulating neurons in a subcortical region of a brain 254 of a mouse 265. As shown in FIG. 2D, implantable piezoelectric ultrasound stimulator device 215 may be surgically inserted and navigated into a brain 254 of a mouse 265 through insertion site 270. Upon application of an alternating voltage over ribbon cable 220, implantable piezoelectric ultrasound stimulator device 215 may generate ultrasound waves and excite nearby neurons. 275 shows a subcortical region of the brain of mouse 265, which is enlarged in 260. As shown in 260, voltages may be applied to electrodes of implantable piezoelectric ultrasound stimulator device 215 over conductive traces 118, 120, thereby causing piezoelectric element 210 to generate ultrasound waves. As shown in FIG. 2D, the generated ultrasound waves may stimulate neurons in the region of the brain near implantable piezoelectric ultrasound stimulator device 215.

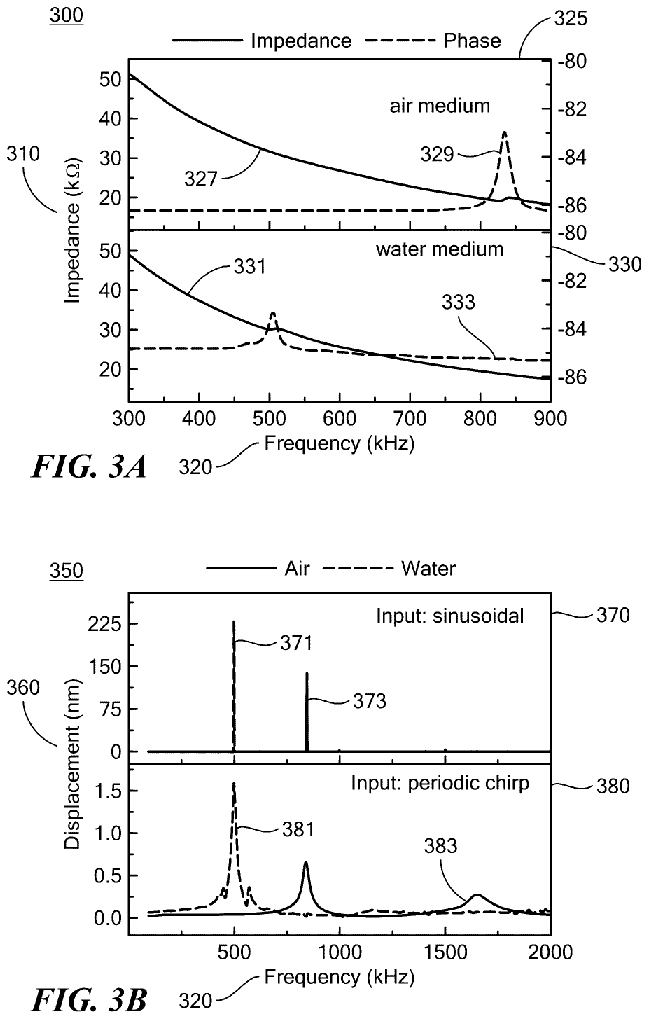

To optimize the device parameters, the electromechanical properties of implantable piezoelectric ultrasound stimulator device 215 were investigated before surgical implantation. Deionized water was used as a representative testing medium due to its similar acoustic properties to brain tissue, and its similar effects in terms of the resonant frequency shift of implantable piezoelectric ultrasound stimulator device 215. The electrical impedance and phase angle spectra of implantable piezoelectric ultrasound stimulator device 215 was measured in air and water, with the results shown in FIG. 3A.

FIG. 3A includes graphs 300 of impedance and phase angle spectra of an example implantable piezoelectric ultrasound stimulator device, showing the resonance frequency in two different mediums. Each of graphs 325, 330 includes a Y-axis 310 representing impedance (in kilohms (k (2), and an X-axis 320 representing frequency (in kilohertz (kHz)).

Graph 325 shows plots of electrical impedance 327 and phase angle 329 over a range of applied voltage frequencies when example implantable piezoelectric ultrasound stimulator device 215 is submerged in air.

Graph 330 shows plots of electrical impedance 331 and phase angle 333 over a range of applied voltage frequencies when example implantable piezoelectric ultrasound stimulator device 215 is submerged in water.

As can be seen from FIG. 3A, the resonance frequency of implantable piezoelectric ultrasound stimulator device 215 when submerged in water has a pronounced but consistent shift from air due to the hydrostatic forces exerted on the flexible biocompatible polymer (e.g., SU-8) surrounding the device and the flexible piezoelectric thin film (e.g., KNN thin film). That is, the resonance frequency in air was measured at 840 kHz, while the resonance frequency in water was measured at 500 kHz.

Similar resonance behavior of implantable piezoelectric ultrasound stimulator device 215 was observed under a scanning laser Doppler vibrometer (LDV). The resonant electromechanical behavior of implantable piezoelectric ultrasound stimulator device 215 was determined with an LDV by applying a periodic chirp excitation voltage from 100 kHz to 2 MHz to the device and measuring the frequency spectrum of the resulting vibrations, with results shown in FIG. 3B.

FIG. 3B includes graphs 350 of displacement of an example implantable piezoelectric ultrasound stimulator device in two different mediums when the applied voltage is a periodic chirp and when the applied voltage is a sinusoidal signal. Each of graphs 370, 380 includes a Y-axis 360 representing displacement (in nm), and an X-axis 320 representing frequency (in kHz).

Graph 380 includes plots showing the frequency spectrum of the vibrations resulting from applying the periodic chirp excitation voltage described above, with plot 383 showing the frequency spectrum when implantable piezoelectric ultrasound stimulator device 215 is submerged in air, and plot 381 showing the frequency spectrum when implantable piezoelectric ultrasound stimulator device 215 is submerged in water.

Once the resonance frequency was determined, a pure sinusoidal signal was applied to implantable piezoelectric ultrasound stimulator device 215 to determine the maximum displacement of the piezoelectric membrane (e.g., piezoelectric film 106). Graph 370 includes plots showing the displacements, with plot 373 showing a displacement of 137 nm at a resonance frequency of 840 kHz (air medium) and plot 371 showing a displacement of 230 nm at a resonance frequency of 500 kHz (water medium) were achieved upon application of a 4 Volt (V) peak-to-peak sinusoidal signal.

The pressure output of a pMUT device may be proportional to the center displacement of the vibration plate, and the center displacement may represent the point of maximum mechanical energy transduction. Implantable piezoelectric ultrasound stimulator devices 215 were fabricated with different cavity sizes (e.g., diameter of cavity 112) to test the effect of varying the cavity size and applied voltage on the displacement of the piezoelectric film (e.g., piezoelectric film 106).

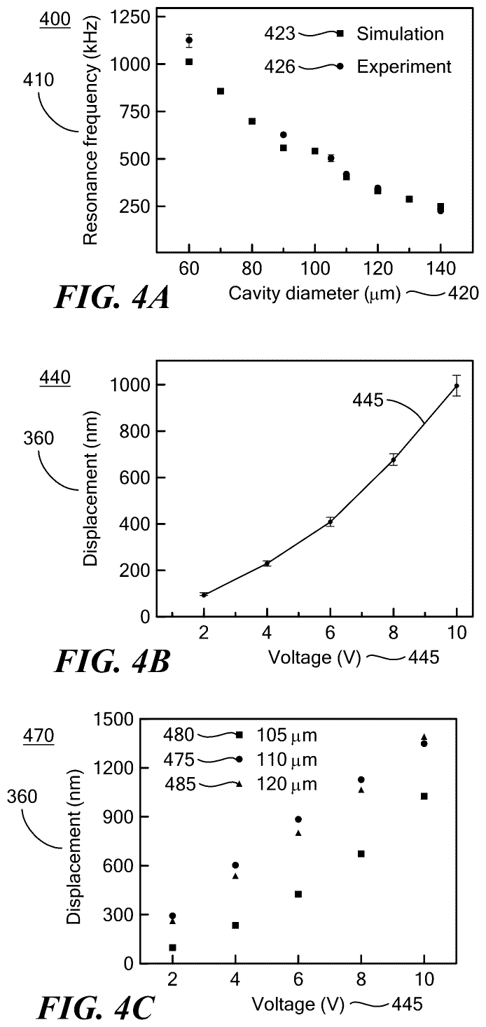

FIG. 4A is a graph 400 showing the simulated and experimentally determined effect of cavity size on the resonant frequency of an example implantable piezoelectric ultrasound stimulator device. Graph 400 includes a Y-axis 410 representing resonance frequency (in kHz), and an X-axis 420 representing cavity diameter (in μm).

Plot 423 includes simulated results showing how the resonance frequency of the implantable piezoelectric ultrasound stimulator device varies as the diameter of the cavity (e.g., cavity 112) varies.

Plot 426 includes experimentally measured results showing how the resonance frequency of the implantable piezoelectric ultrasound stimulator device varies as the diameter of the cavity (e.g., cavity 112) varies.

As can be seen from plots 423 and 426, the resonance frequency decreases with an increase in the diameter of the cavity.

The most significant modulation of neurons may occur with the application of ultrasound at frequencies less than 1 MHz.

FIG. 4B is a graph 440 of displacement as a function of input voltage for an example implantable piezoelectric ultrasound stimulator device 215 with cavity size of 105 μm and having a resonant frequency of 500 kHz. Graph 440 includes a Y-axis 360 representing displacement (in nm), and an X-axis 445 representing applied voltage (in V).

Plot 445 shows how the peak displacement of the implantable piezoelectric ultrasound stimulator device varies as the applied voltage varies from 2V peak-to-peak to 10V peak-to-peak. Thus, as shown in graph 440, the applied voltage affects the peak displacement of the implantable piezoelectric ultrasound stimulator device.

FIG. 4C is a graph 470 showing the measured displacement of example implantable piezoelectric ultrasound stimulator devices having different cavity sizes and applied voltages. Graph 470 includes a Y-axis 360 representing displacement (in nm), and an X-axis 445 (representing voltage (in V).

Graph 470 includes plots 475, 480, and 485 showing how peak displacement of the implantable piezoelectric ultrasound stimulator device may vary based on cavity size and applied voltage. Plot 480 represents a cavity size of 105 μm, plot 475 represents a cavity size of 110 μm, and plot 485 represents a cavity size of 120 μm. As shown in graph 470, an increase in cavity size (e.g., increase in diameter of cavity 112) may lead to higher displacement of the implantable piezoelectric ultrasound stimulator device, which may be attributed to a reduced stiffness of the piezoelectric film (e.g., piezoelectric film 106) with the increased cavity size.

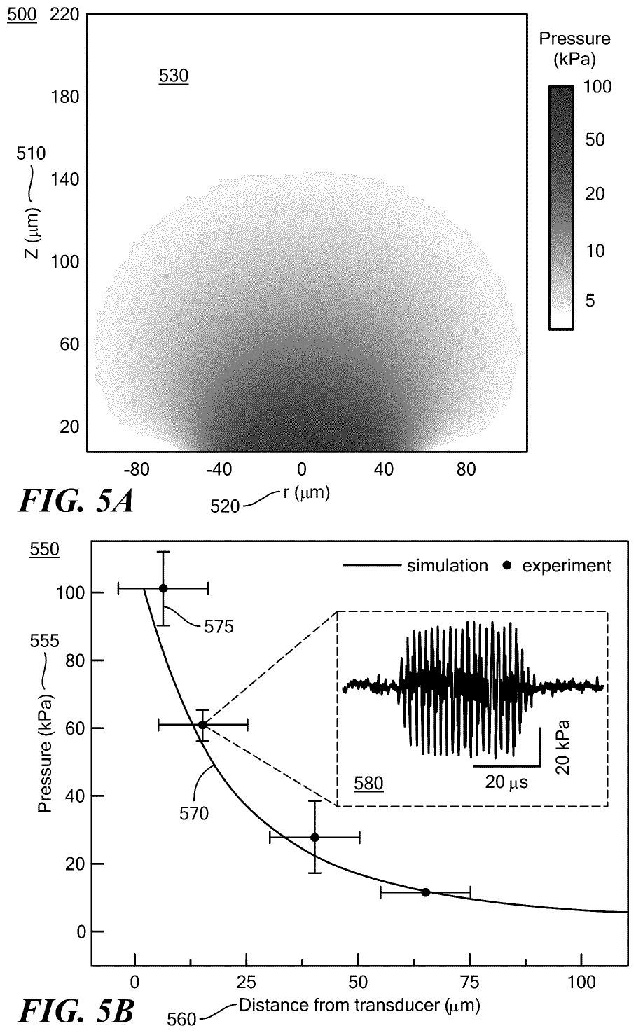

FIG. 5A is a graph 500 of a simulated acoustic pressure profile of an example implantable piezoelectric ultrasound stimulator device. Graph 500 includes a Y-axis 510 representing a height Z (in μm) in a direction from the top layer (e.g., top layer 102) of the implantable piezoelectric ultrasound stimulator device, and an X-axis 520 representing a radial distance r (in μm) in a direction perpendicular to the direction from the top layer.

Graph 500 includes a plot 530 that shows that maximum pressure adjacent the implantable piezoelectric ultrasound stimulator device may be as high as 100 kPa, and may decrease gradually following a spherical pressure distribution.

FIG. 5B is a graph 550 comparing simulated and experimentally measured pressures at different distances from an example implantable piezoelectric ultrasound stimulator device. Graph 550 includes a Y-axis 555 representing pressure (in kPa), and an X-axis 560 representing a distance from a top layer (e.g., top layer 102) of an implantable piezoelectric ultrasound stimulator device.

Plot 570 shows simulated pressure as a distance from a top layer of the implantable piezoelectric ultrasound stimulator device varies. Plot 575 shows experimentally measured pressures as a distance from a top layer of the implantable piezoelectric ultrasound stimulator device varies. Inset 580 shows that a maximum pressure of approximately 59.2 kPa was measured approximately 15 μm away from the implantable piezoelectric ultrasound stimulator device. As can be seen from plots 570 and 575, a pressure of approximately 100 kPa is measured adjacent to the implantable piezoelectric ultrasound stimulator device.

Ultrasound generation from an implantable piezoelectric ultrasound stimulator device may result in a temperature rise in the surrounding medium due to the intrinsic heating of the piezoelectric film (e.g., piezoelectric film 106) and resistive losses.

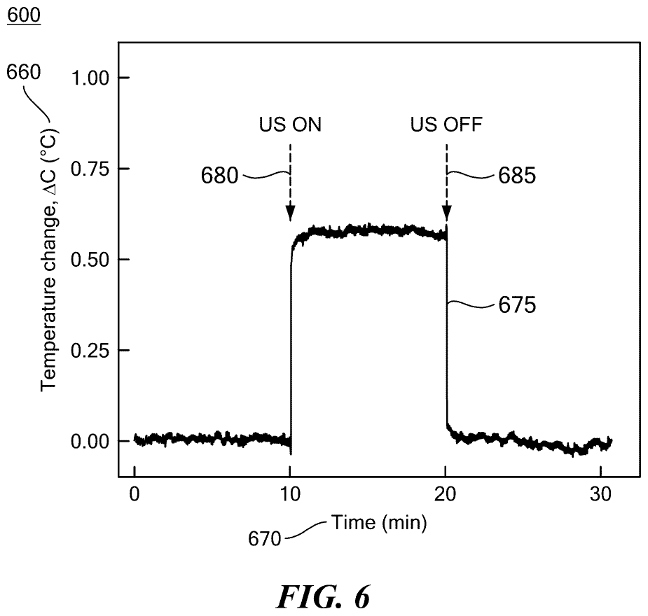

FIG. 6 is a graph 600 showing a temperature change in a water medium when an example implantable piezoelectric ultrasound stimulator device is operating. Graph 600 includes a Y-axis 660 representing temperature change (in ° C.), and an X-axis 670 representing time (in minutes).

Plot 675 shows the temperature change in a water medium (with a 22.5° C. baseline temperature) during ultrasound application, with the thermometer measuring the temperature change positioned 15 μm from the top layer (e.g., top layer 102) of the implantable piezoelectric ultrasound stimulator device. 680 shows when the ultrasound stimulation based on an applied sinusoidal voltage of 20V peak-to-peak was started, and 685 shows when it was ended. As shown in FIG. 6, a ten minute application of the continuous sinusoidal signal at an input voltage of 20V peak-to-peak gave rise to an increase in temperature of only 0.6° C., which is much less than the threshold of temperature-evoked neuromodulation. Thermogenic effects may be further reduced without affecting peak pressures by applying a pulsed voltage, rather than a continuous voltage.

To test that an example implantable piezoelectric ultrasound stimulator device will remain functional over a long period of use within a harsh biological environment, the durability of an implantable piezoelectric ultrasound stimulator device was tested by performing an accelerated aging test in a phosphate-buffered saline (PBS) solution at an elevated temperature of 75° C. for seven days. Microscopic images of the implantable piezoelectric ultrasound stimulator device showed that there was minimal damage visually to the device after seven days of continuous exposure to PBS at 75° C.

To confirm that the implantable piezoelectric ultrasound stimulator device remains fully functional, the displacement of the implantable piezoelectric ultrasound stimulator device was measured before and after the seven days of the aging test. The displacement of the implantable piezoelectric ultrasound stimulator device was found to degrade by only 2.4% over the seven days of the aging test.

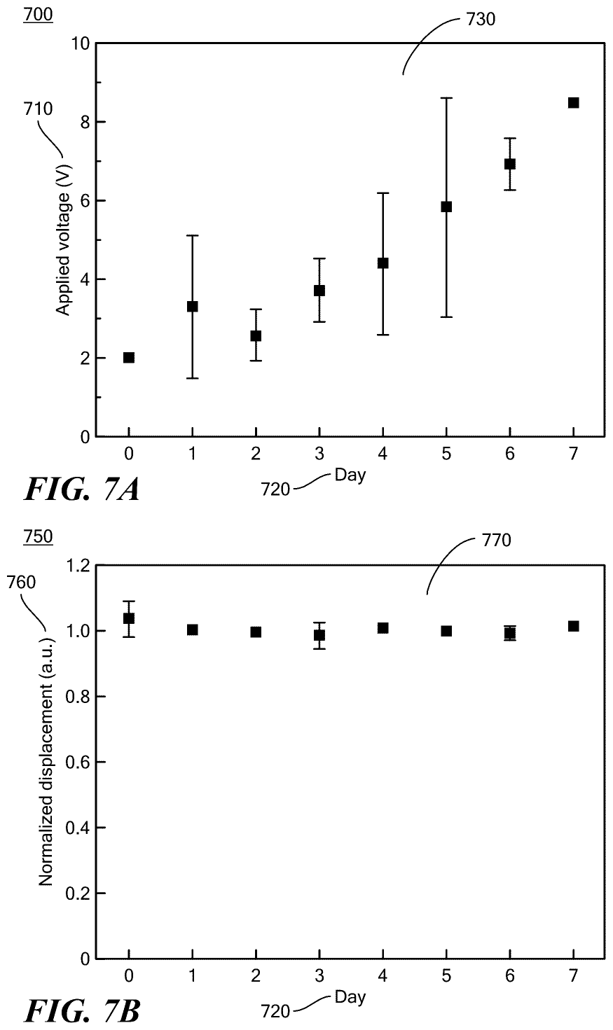

In order to simulate use of the implantable piezoelectric ultrasound stimulator device as a chronic device, the accelerated aging test was repeated with an additional application of stressor where for a daily period of 10 minutes the device was turned on and generating ultrasound waves. The voltage necessary each day to maintain the performance of the device was recorded, with the results shown in FIG. 7A.

FIG. 7A is a graph 700 showing applied voltages to maintain a steady displacement of an example implantable piezoelectric ultrasound stimulator device over days of the accelerated aging test. Graph 700 includes a Y-axis 710 representing the applied voltage (in V), and an X-axis 720 representing day of the accelerated aging test.

FIG. 7B is a graph 750 showing the normalized displacement of an example implantable piezoelectric ultrasound stimulator device over days of an aging test when the applied voltages of FIG. 7A were used. Graph 750 includes a Y-axis 760 representing normal displacement of the implantable piezoelectric ultrasound stimulator device (in arbitrary units (a.u.)), and an X-axis 720 representing day of the accelerated aging test.

Plot 730 of graph 700 shows the applied voltage necessary on each day to maintain the performance (i.e., displacement) of the implantable piezoelectric ultrasound stimulator device. As can be seen from graph 730, the applied voltage during the accelerated aging test was adapted to compensate for the loss of vibrational amplitude of the implantable piezoelectric ultrasound stimulator device over the course of the seven days of the accelerated aging test.

Plot 770 of graph 750 shows that, by applying the adaptive voltages of graph 700, the normalized displacement of the implantable piezoelectric ultrasound stimulator device was maintained over the seven days of the accelerated aging test.

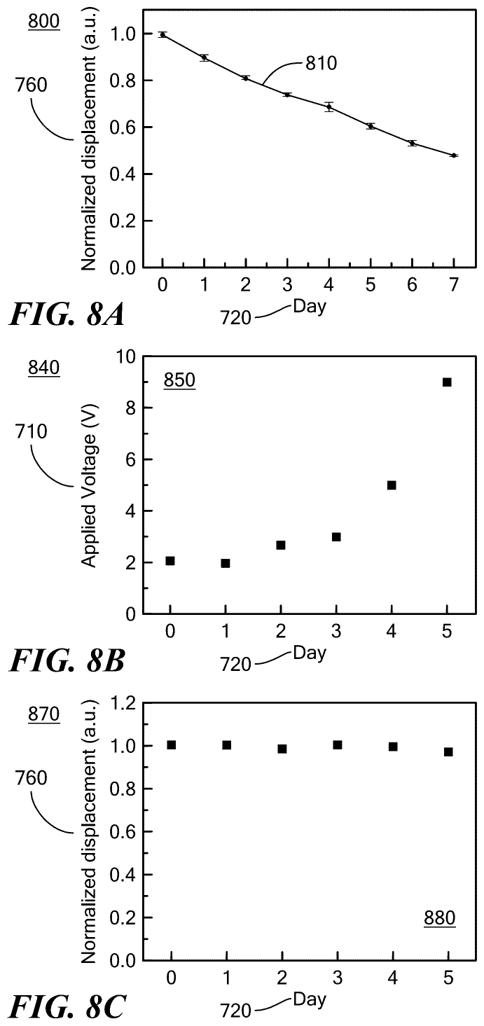

The durability of the example implantable piezoelectric ultrasound stimulator device was further tested with a fatigue test, during which a continuous sinusoidal signal of 500 kHz at 10V peak-to-peak was applied continuously for seven days and a corresponding displacement of the device was recorded in a water medium. The implantable piezoelectric ultrasound stimulator device was exposed to 302.4 billion cycles of an applied sine wave voltage over the course of seven days. The results are shown in FIG. 8A.

FIG. 8A is a graph 800 showing the normalized displacement of an example implantable piezoelectric ultrasound stimulator device over days of a fatigue test. Graph 800 includes a Y-axis 760 representing normalized displacement of the implantable piezoelectric ultrasound stimulator device (in arbitrary units (a.u.)), and an X-axis 720 representing day of the fatigue test.

As shown by plot 810 in graph 800, after seven days of the fatigue test, the implantable piezoelectric ultrasound stimulator device had approximately 40% lower amplitude of displacement, due to degradation of the piezoelectric film (e.g., piezoelectric film 106) after the extended electric cycling (i.e., 302.4 billion cycles of an applied sine wave voltage over the course of seven days). However, like with the accelerated aging test, compensation for the reduction in performance loss due to fatigue may be achieved by the application of an adaptive voltage, as shown in FIGS. 8B and 8C.

FIG. 8B is a graph 840 showing the applied voltage used to maintain a steady displacement of an example implantable piezoelectric ultrasound stimulator device over days of a fatigue test. Graph 840 includes a Y-axis 710 representing an applied voltage (in V), and an X-axis 720 representing day of the fatigue test.

FIG. 8C is a graph 870 showing the normalized displacement of an example implantable piezoelectric ultrasound stimulator device over days of the fatigue test when the applied voltages of graph 840 were used. Graph 870 includes a Y-axis 760 representing a normalized displacement of the implantable piezoelectric ultrasound stimulator device (in arbitrary units (a.u.)), and an X-axis 720 representing day of the fatigue test.

Plot 850 of graph 840 shows the applied voltage necessary on each day of the fatigue test to maintain the performance (i.e., displacement) of the implantable piezoelectric ultrasound stimulator device. As can be seen from graph 840, the applied voltage during the fatigue test was adapted to compensate for the loss of displacement of the implantable piezoelectric ultrasound stimulator device over the course of five days of the fatigue test.

Plot 880 of graph 870 shows that, by applying the adaptive voltages of graph 840, the normalized displacement of the implantable piezoelectric ultrasound stimulator device was maintained over five days of the fatigue test.

An example implantable piezoelectric ultrasound stimulator device was also tested for biocompatibility of its constituent materials with a cell viability test. Cortical tissues from embryonic mice were harvested and dissociated cells were seeded on a glass-bottomed dish containing a fixed implantable piezoelectric ultrasound stimulator device. The dissociated cells were allowed to differentiate into cultured primary neurons for a period of 10 days in the presence of the implantable piezoelectric ultrasound stimulator device. Cell densities across six plates (3 for control and 3 with an implantable piezoelectric ultrasound stimulator device) were assessed and found to stabilize after a cell medium change on day 3. Neurons on both the control plates and the plates with the implantable piezoelectric ultrasound stimulator devices differentiated neurites normally and developed neurites stably after the 10-day period.

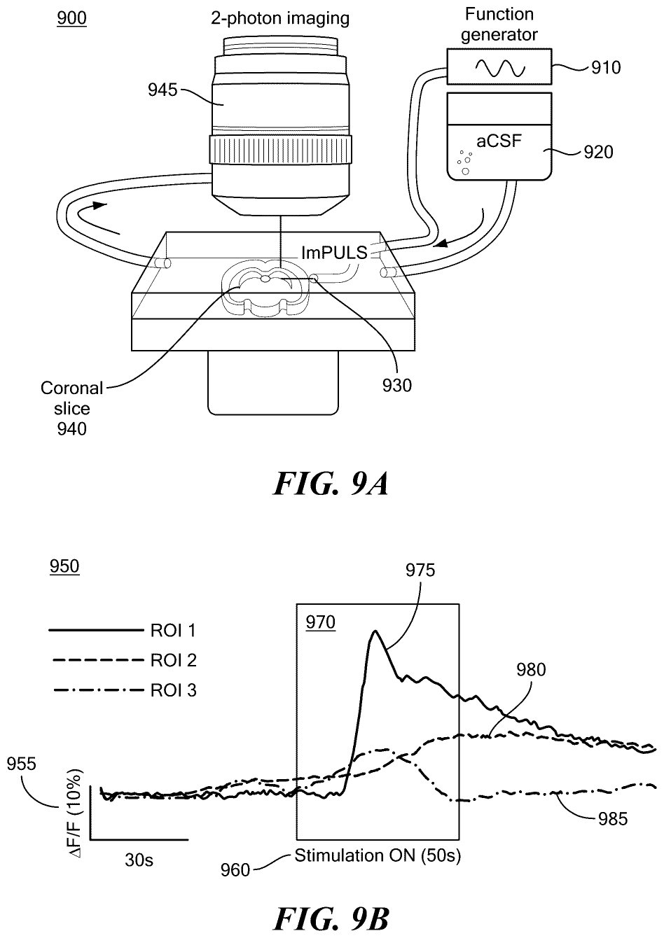

FIG. 9A is a diagram 900 of a two-photon imaging setup with a coronal hippocampal slice 940 and an example implantable piezoelectric ultrasound stimulator device 930 under magnification (by 2-photon imaging apparatus 945), where artificial cerebrospinal fluid (aCSF) 920 was circulated throughout a bath and a function generator 910 connected to the leads outside the bath provided excitation for ultrasonic stimulation.

Using the setup of diagram 900, the potential of an example implantable piezoelectric ultrasound stimulator device 930 to stimulate healthy neurons in coronal hippocampal slice 940 was evaluated with 2-photon imaging apparatus 945. Hippocampal neurons expressed the genetically encoded calcium indicator GCaMP7F to report neural activity during ultrasonic stimulation. Neurons in the dentate gyrus were targeted for stimulation. After a 60 second baseline period, a sinusoidal pulse (500 kHz, 10V peak-to-peak amplitude, with 1.5 kHz repetition frequency (PRF) and 50% duty factor) was used to stimulate neurons for 50 seconds. After the stimulation ended, population activity was captured for another 60 seconds. Several neurons in the field of view were activated during ultrasound stimulation.

FIG. 9B is a graph 950 of fluorescence change in three different regions of interest over time using the setup of FIG. 9A. Graph 950 includes a Y-axis 955 representing a change in fluorescence, and an X-axis 960 representing time.

970 in graph 950 represents the period of time (i.e., 50 s) in which the implantable piezoelectric ultrasound stimulator device was turned on and generating ultrasound waves. As shown by plot 975 in graph 950, marked neurons in region of interest 1 (ROI 1) exhibited a 30% change in fluorescence approximately 15 s after stimulation began and reached a maximum intensity approximately 9 seconds after the initial rise. Marked neurons in region of interest 2 (ROI 2) and in region of interest 3 (ROI 3) belong to the same local cluster of neurons and showed smaller changes in activity during stimulation, as shown by plots 980 and 985, respectively, of graph 950. The delay in neural activation may have been a result of the high amounts of dissolved gasses in the artificial cerebrospinal fluid (aCSF), which may lower cavitation thresholds and cause ultrasound energy from small sources to be absorbed into the medium rather than into the tissue. These results confirm that the implantable piezoelectric ultrasound stimulator device can activate local hippocampal neurons.

Another test was performed to test the potency of the example implantable piezoelectric ultrasound stimulator device to activate cellular ensembles in vivo in mice. To do so, an implantable piezoelectric ultrasound stimulator device was surgically implanted into the hippocampus (here the hippocampus of a mouse), which is a subcortical brain region that is essential for learning and memory across mammalian species. More specifically, the implantable piezoelectric ultrasound stimulator device was targeted to the dorsal CA1 (dCA1) layer of the hippocampal formation to test the efficacy of different stimulation protocols. Relative levels of cFos in dCA1, which is a widely used marker of recent neuronal activity, were used to measure the extent of neural activation resulting from different bouts of stimulation from the implantable piezoelectric ultrasound stimulator device with the mouse under anesthesia.

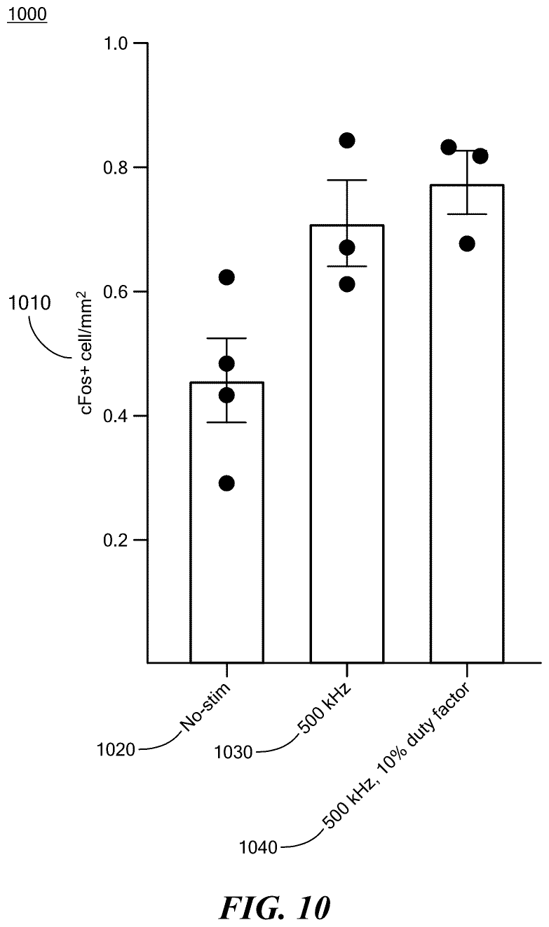

Compared to a no-stimulation (control) group, all stimulation parameters increased the levels of activity-dependent gene cFos on average. Some results are shown in FIG. 10.

FIG. 10 is a graph 1000 showing some results of the experiment measuring cFos+protein in the dorsal CA1 (dCA1) layer of the hippocampal formation after stimulation with an example implantable piezoelectric ultrasound stimulator device operating at a frequency of 500 kilohertz (kHz), after stimulation with an example implantable piezoelectric ultrasound stimulator device operating at a frequency of 500 kHz with a 10% duty factor, and without stimulation. Graph 1000 includes a Y-axis 1010 representing cFos cell activity levels per square millimeter of tissue, and an X-axis showing different stimulation approaches used in the test.

As shown in graph 1000, applying no stimulation (1020) with the implantable piezoelectric ultrasound stimulator device resulted in a little over 0.4 cFos cell expression per square millimeter, applying stimulation at an applied voltage frequency of 500 kHz (1030) resulted in approximately 0.7 cFos cell expression per square millimeter, and applying stimulation at an applied voltage frequency of 500 kHz with a 10% duty factor (1040) resulted in approximately 0.8 cFos cell expression per square millimeter. That is, an approximately 2-fold increase in cFos expression was observed following stimulation with a 500 kHz 10% duty factor applied voltage as compared to the control where no stimulation was applied. This suggests that stimulation using an applied voltage frequency of 500 kHz with a 10% duty factor was sufficient to activate large populations of cells in the dCA1 layer of the mouse hippocampus.

Stimulation of the dCA1 layer in mice by an implantable piezoelectric ultrasound stimulator device was also tested 14 days after the implantable piezoelectric ultrasound stimulator device was implanted in the mice. The 500 kHz, 10% duty factor stimulation group showed a significant increase in average cFos+/DAPI+ in the dCA1 layer using an unpaired t-test. These results indicate that stimulation with the implantable piezoelectric ultrasound stimulator device was sufficient to elicit neuronal activation following chronic implantation, which suggests that the implantable piezoelectric ultrasound stimulator device is functionally viable on more chronic timescales.

Utility of ultrasound stimulation using the implantable piezoelectric ultrasound stimulator device was also tested for functional modulation of neurotransmission in vivo using an anesthetized mouse. Dopaminergic (DA) neurons of the substantia nigra pars compacta (SNc) innervate the dorsal striatum (DS) to form the canonical nigrostriatal dopamine pathway, which is a circuit crucial for movement and reinforcement in the mammalian brain. Furthermore, excitatory stimulation of dopaminergic transmission has therapeutic implications in the treatment of Parkinson's Disease (PD) and memory disorders. The tests sought to modulate nigrostriatal DA through ultrasound stimulation using the implantable piezoelectric ultrasound stimulator device of the SNc using an anesthetized mouse.

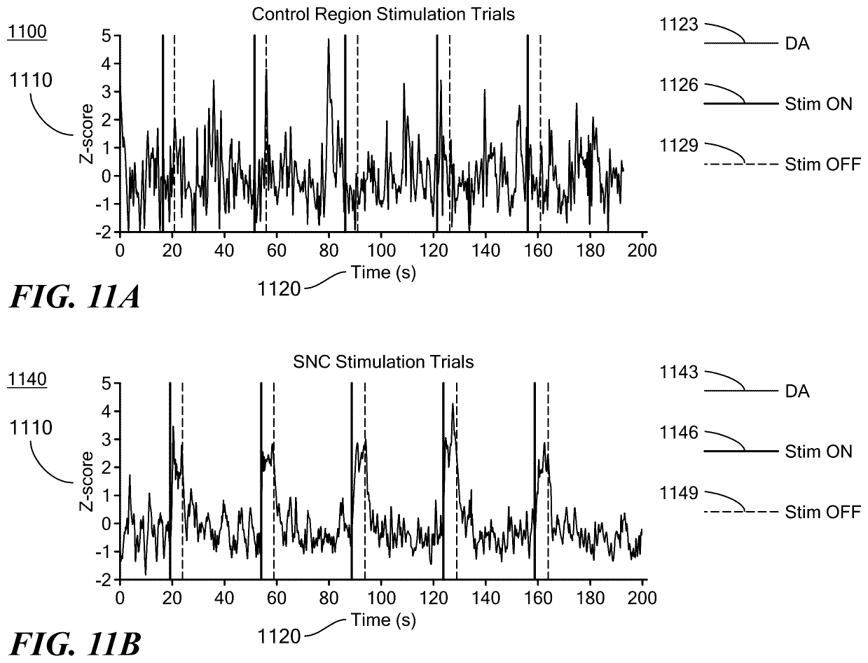

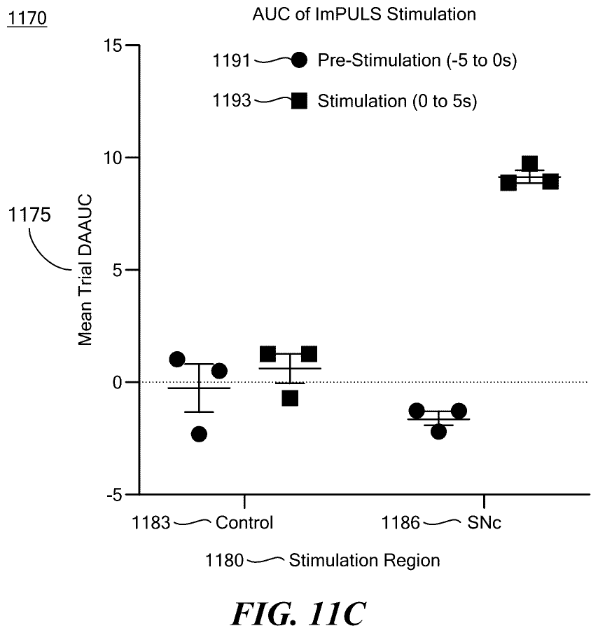

Implantation of the implantable piezoelectric ultrasound stimulator device was targeted to the anterior SNc with a lateral stimulation direction. Fiber photometry recordings of extra-cellular DA release in the DS were recorded using a genetically encoded DA sensor, GRAB-DA2m. Pulsed (PRF 1500 Hz, 50% duty factor) stimulation of the SNc for 5 seconds (514 kHz, 10V peak-to-peak) elicited robust, time-locked increases in striatal DA release. Control stimulation trials, in which tissue approximately 200 μm dorsal to the SNc was stimulated, failed to alter DA2m fluorescence. Therefore, at least in the areas of tissue inferior to the implantable piezoelectric ultrasound stimulator device, stimulation did not reach beyond 100 μm. By contrast, a mean increase in DA2m fluorescence between 2 and 3 Z-scores was observed through the duration of SNc stimulation using the implantable piezoelectric ultrasound stimulator device. These results are shown in FIGS. 11A and 11B.

FIG. 11A is a graph 1100 showing the results of an experiment recording Z-score DA2m fluorescence in a control region of a brain where stimulation from an example implantable piezoelectric ultrasound stimulator device is turned on and off. Graph 1100 includes a Y-axis 1110 representing Z-score, and an X-axis 1120 representing time. Lines 1126 show times at which stimulation by the implantable piezoelectric ultrasound stimulator device is started, and lines 1129 show times at which stimulation by the implantable piezoelectric ultrasound stimulator device is ended.

FIG. 11B is a graph 1140 showing the results of an experiment recording Z-score DA2m fluorescence in the substantia nigra pars compacta (SNc) region of a brain where stimulation from an example implantable piezoelectric ultrasound stimulator device is turned on and off. Graph 1140 includes a Y-axis 1110 representing Z-score, and an X-axis 1120 representing time. Lines 1146 show times at which stimulation by the implantable piezoelectric ultrasound stimulator device is started, and lines 1149 show times at which stimulation by the implantable piezoelectric ultrasound stimulator device is ended.

Plot 1123 of graph 1100 shows the DA2m fluorescence recorded in the control region of the brain over time, while plot 1143 of graph 1140 shows the DA2m fluorescence recorded in the SNc region of the brain over time. As can be seen by comparing plots 1123 and 1143, reduced magnitude and variability of DA signaling is pronounced between stimulation of the control region of the brain as compared to stimulation of the SNc region of the brain.

The mean area under the curve (AUC) was calculated across stimulation trials from the 5 second baseline period before stimulation and the 5 second period during stimulation of both the SNc and control regions of the brain. The results are shown in FIG. 11C.

FIG. 11C is a graph 1170 showing the area under the curve of FIGS. 10A and 10B in the 5 second baseline period before stimulation and the 5 second period during stimulation and in the SNc and control regions of the brain using the example implantable piezoelectric ultrasound stimulator device. Graph 1170 includes a Y-axis 1175 representing the mean area under the curve, and an X-axis showing the stimulation region of the brain.

Plot 1191 shows the mean AUC in the 5 second baseline period before stimulation (pre-stimulation (−5 to 0 s)) and plot 1193 shows the mean AUC in the 5 second period during stimulation (stimulation (0 to 5 s)). 1183 represents the results for the control region of the brain and 1186 represents the results for the SNc region of the brain.

As shown in graph 1170, mean AUC of DA fluorescence during the SNc stimulation period was significantly different from the mean AUC of DA fluorescence of the SNc pre-stimulation period. Moreover, graph 1170 shows that stimulation of the SNc region was significantly different than stimulation of the control region.

The results shown in graph 1170 suggest that the implantable piezoelectric ultrasound stimulator device evokes robust nigrostriatal DA release in a spatially localized manner, given that stimulation of tissue only 200 μm above the SNc failed to alter DA release.

The results described with respect to FIGS. 9A-11C demonstrate that spatially localized brain ultrasound stimulation using the implantable piezoelectric ultrasound stimulator device is capable of modulating neurotransmission in vivo and ex vivo. Thus, the implantable piezoelectric ultrasound stimulator device may be a useful device for use in therapeutic or investigative applications.

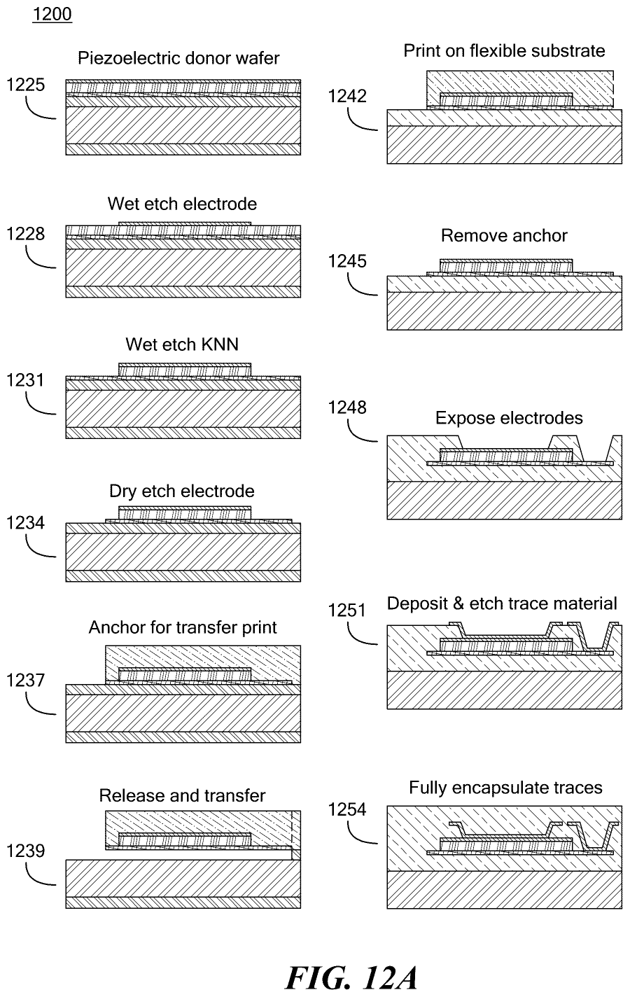

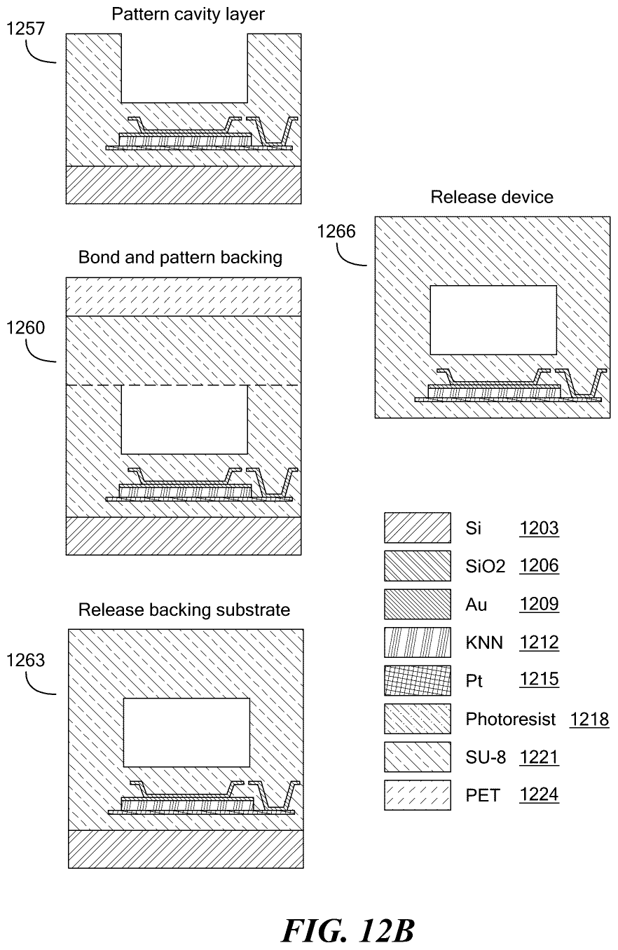

FIGS. 12A and 12B are diagrams of a process 1200 for making an example implantable piezoelectric ultrasound stimulator device. FIG. 12A shows a portion of process 1200, and FIG. 12B shows another portion of process 1200. Process 1200 may be used, for example, to make an implantable piezoelectric ultrasound stimulator device as shown and/or discussed with respect to FIGS. 1A-11C or 13-15.

As shown in FIG. 12A, process 1200 may begin in 1225 with a stack of layers on top of a first substrate. In the example shown in FIG. 12A, the first substrate may comprise a first layer of silicon dioxide (SiO₂), a layer of silicon (Si), and a second layer of SiO₂, though the disclosure is not so limited. The stack of layers may comprise at least a first electrode layer in contact with the top of the first substrate and comprising at least one first electrode (e.g., top electrode 104) to be formed, a second electrode layer comprising at least one second electrode (e.g., bottom electrode 110) to be formed, and a piezoelectric layer comprising at least one piezoelectric film (e.g., piezoelectric film 106) to be formed. That is, the stack of layers during process 1200 may be inverted from the way materials are stacked in the FIGS. 1A, 1B. In some embodiments, the stack of layers comprises a layer of zinc oxide (ZnO). In some embodiments, the layer of ZnO may help to align the lattice structures of the materials, in order to vertically align the grain structure of the layers that end up on top (e.g., platinum (Pt) and KNN) during later annealing steps. In some embodiments, the stack of layers comprises a layer of platinum (Pt) as the first electrode layer, a layer of chromium (Cr) and a layer of gold (Au) as the second electrode layer, and a layer of KNN as the piezoelectric layer. The layers may be provided on a donor wafer, though the disclosure is not so limited.

In some embodiments, the first layer of SiO₂ in the first substrate may be 500 nm thick, the layer of Si in the first substrate may be 610 μm thick, and the second layer of SiO₂ in the first substrate may be 500 nm thick, though the disclosure is not so limited. In some embodiments, the layer of ZnO in the stack of layers may be 30 nm thick, though the disclosure is not so limited. In some embodiments, the layer of Pt in the stack of layers may be 200 nm thick, the layer of KNN in the stack of layers may be 1 μm thick, the layer of Cr in the stack of layers may be 10 nm thick, and the layer of Au in the stack of layers may be 150 nm thick, though the disclosure is not so limited.

In some embodiments, the stack of layers of 1225 may be processed in preparation for transfer printing. For example, an etching pattern may be formed on the stack of layers for etching the electrodes and piezoelectric film. In some embodiments, contact photolithography with spin-coated positive i-line photoresist (PR) of 15 μm thickness may be used to define wet-etching patterns for top (e.g., top electrode 104) and bottom (e.g., bottom electrode 110) electrodes and for the piezoelectric film (e.g., piezoelectric film 106).

In 1228, the bottom electrode(s) (e.g., bottom electrode(s) 110) may be etched. For example, in some embodiments, Au and Cr layers of the bottom electrode(s) (e.g., bottom electrode 110) may be etched with gold etchant and CR-7 chrome etchant, respectively.

In 1231, the KNN may be etched. In some embodiments, KNN may be defined in 1231 using the same photoresist mask and wet etching as in 1228. In some embodiments, the KNN may be etched with a 49% hydrofluoric acid. This acidic wet-etching chemistry for KNN patterning may achieve faster etch rates of up to an etch rate of 100 nm/min, which may mediate the undercut effect (discussed later herein) and which may allow for denser and smaller transducer pitches.

In some embodiments, the photoresist may then be stripped. For example, the photoresist may be stripped in a 120° C. heated N-methyl-2-pyrrolidone (NMP)-based stripper.

In 1234, a pattern for the top electrode(s) (e.g., top electrode(s) 104) may be defined and the top electrode(s) may be etched. For example, the pattern may be defined with a photoresist etch mask. The top electrode(s) may then be dry-etched using a reactive-ion etching (RIE) process. In the example of FIG. 12A, both ZnO and Pt may be dry-etched using the RIE process. In some embodiments, the RIE process may flow Ar/O₂ (95%/5%) and power of 500 Watts (W).

In 1237, the stack of layers and at least a portion of the first substrate may be coated with an anchor material. In some embodiments, the anchor material may comprise a photoresist layer. In some embodiments, the photoresist layer may be designed to both preserve element-to-element alignment and to serve as a mechanical support during an undercutting, release, and transfer printing process.

In 1239, the stack of layers may be undercut. For example, the stack of layers may be undercut by removing at least a portion of the first substrate. In some embodiments, the stack of layers may be undercut by removing at least a portion of the second layer of SiO₂ of the first substrate. In some embodiments, the stack of layers may be undercut using a diluted hydrogen fluoride (HF) solution with a weight ratio of 49%/60% HF/deionized (DI) water. In some embodiments, the structure of 1239 may be rinsed. In some embodiments, the stack of layers and at least a portion of the anchor material may then be removed from the remaining portion of the first substrate. For example, in some embodiments, the stack of layers and at least a portion of the anchor material may be delaminated from the rest of first substrate (e.g., from the rest of the second layer of SiO₂ of the first substrate) using a thermal release tape.

In some embodiments, the addition of the anchor material (e.g., photoresist) may be crucial to maintaining the relative spacing of elements during the undercutting process. The anchor material may provide an anchor designed to extend the processing window and to mechanically support the pattern in the stack of layers with the anchor material (e.g., photoresist) to ensure high yield in removing the stack of layers from the first substrate (e.g., via transfer to thermal tape). Once transferred to the thermal tape, the patterns of the stack of layers may be prepared to be printed on a layer of flexible biocompatible polymer (e.g., SU-8), as will be further discussed herein.

A separate, second substrate (e.g., Si substrate) wafer may be prepared and coated with a release layer (e.g., Omnicoat release layer), which may then be coated with a first layer of biocompatible polymer (e.g., SU-8). In some embodiments, the first layer of the biocompatible polymer may be 0.5 μm thick, though the disclosure is not so limited. In some embodiments, the structure comprising the substrate, release layer, and first layer of biocompatible polymer may then be soft-baked and flood-exposed (e.g., for 2 seconds with 365 nm ultraviolet (UV) light).

In 1242, the stack of layers and the at least a portion of the anchor material coated on the stack of layers may be pressed onto the first layer of the biocompatible polymer (e.g., SU-8), which as discussed may be positioned atop a release layer and a second substrate. For example, the pattern of the stack of layers and the at least a portion of the anchor material on the thermal tape may be pressed on the first layer of the biocompatible polymer (e.g., SU-8). In some embodiments, the pattern of the stack of layers and the at least a portion of the anchor material may be left on a hot plate to slowly heat up to a release temperature during the post-exposure bake of the first layer of the biocompatible polymer. In some embodiments, at the release temperature, the tape may be gently removed (e.g., with tweezers or with a machine). In some embodiments, the first layer of biocompatible polymer may later become the top layer (e.g., top layer 102) of the implantable piezoelectric ultrasound stimulator device.

In 1245, the at least a portion of the anchor material (e.g., photoresist) may be removed from the pattern of the stack of layers. For example, the at least a portion of the anchor material may be removed by spraying acetone and 2-propanol on the structure of 1242.

In 1248, a second layer of biocompatible polymer (e.g., SU-8) may be coated onto the stack of layers and onto the first layer of biocompatible polymer (e.g., SU-8), leaving openings (e.g., via patterning) to the top and bottom electrodes for metallization and metal interconnects to bond pads. For example, the structure of 1245 may be subjected to a short RIE O₂ plasma treatment, and then a second layer of biocompatible polymer (e.g., SU-8) may be spun on to the stack of layers and onto the first layer of biocompatible polymer, leaving openings to the top and bottom electrodes for metallization and metal interconnects to bond pads. In some embodiments, the structure of 1248 may then be hard-baked (e.g., at 120° C.) to ensure proper adhesion between layers and to reduce the angle of the sidewalls around the metal contact openings to ensure proper connectivity after metallization.

In 1251, metal interconnects and bond pads may be deposited and etched. The metal interconnects and bond pads may be later used to connect the at least one first electrode (e.g., top electrode 104) and the at least one second electrode (e.g., bottom electrode 110) to external circuitry. In some embodiments, Cr and Au may be sputter deposited over the top of structure 1248 and then patterned (e.g., with a photoresist mask) for wet etching to define the metal interconnects and larger bond pads that will allow the device to be connected to external instrumentation. In some embodiments, 10 nm of Cr and 200 nm of Au may be sputter deposited over the top of structure 1248 and then patterned, though the disclosure is not so limited.

In some embodiments, the structure of 1251 may then be rinsed, and then the resistivity of the resulting surface may be checked to ensure complete etching of the conductive material. In some embodiments, the resulting structure may then be stripped of photoresist, cleaned, dried (e.g., with a N₂ spray gun), and treated with O₂ plasma preceding deposition of the next layer of biocompatible material.

In 1254, a third layer of biocompatible polymer (e.g., SU-8) may be coated onto the metal interconnects and the second layer of biocompatible polymer (e.g., SU-8). In some embodiments, a 0.5-0.8 μm thick layer of the biocompatible polymer (e.g., SU-8) may be coated onto the metal interconnects and the second layer of biocompatible polymer as the third layer of biocompatible polymer. In some embodiments, the third layer of biocompatible polymer may then be exposed and cured to complete insulation and encapsulation of the electrically active elements of the implantable piezoelectric ultrasound stimulator device. In some embodiments, the second layer of biocompatible polymer and third layer of biocompatible polymer may later become the membrane layer (e.g., membrane layer 108) of the implantable piezoelectric ultrasound stimulator device.

Turning to FIG. 12B, in 1257 a cavity (e.g., cavity 112) may be patterned onto the third layer of biocompatible polymer, and a fourth layer of biocompatible polymer (e.g., SU-8) (e.g., cavity layer 114) may be coated onto the third layer of biocompatible polymer, except where the cavity was patterned. In some embodiments, the fourth layer of biocompatible polymer (e.g., SU-8) may be a thicker mechanical layer (e.g., ˜15 μm). In some embodiments, the fourth layer of biocompatible polymer may be designed to control the resonant frequency of the implantable piezoelectric ultrasound stimulator device (see, e.g., FIGS. 4A, 4C). In some embodiments, the fourth layer of biocompatible polymer may later become the cavity layer (e.g., cavity layer 114) of the implantable piezoelectric ultrasound stimulator device.

In some embodiments, a fifth layer of biocompatible polymer (e.g., SU-8) may be prepared on a third substrate. For example, a thin layer of polyvinyl alcohol (PVA) (e.g., 4 wt %) may be spin-coated to a polyethylene terephthalate (PET) film (e.g., at 3000 revolutions per minute (rpm) for 45 seconds), followed by an annealing process (e.g., 10 minutes at 110° C.), though the disclosure is not so limited. This PVA layer may act as a sacrificial layer to aid in the delamination of the PET backing during development. In some embodiments, following a short RIE O₂ plasma treatment, the fifth layer of biocompatible polymer (e.g., SU-8) may be spun onto the PVA layer. In some embodiments, the fifth layer of biocompatible polymer may be spun (e.g., at 6000 rpm for 45 seconds). In some embodiments, the PET film, PVA layer, and fifth layer of biocompatible polymer may then be soft-baked (e.g., for 5 minutes at 65° C. followed by 5 minutes at 95° C.) in order to achieve an appropriate surface of the fifth layer of biocompatible polymer for bonding.

In 1260, the fourth layer of the biocompatible polymer may be bonded to a fifth layer of biocompatible polymer (e.g., SU-8) on the third substrate. For example, the fourth layer of the biocompatible polymer may be bonded to the fifth layer of the biocompatible polymer (e.g., SU-8) on the PVA of the third substrate and pressed gently. In some embodiments, the structure of 1260 may be exposed through the PET film to seal the fourth layer of biocompatible material (e.g., cavity layer 114) to the fifth layer of biocompatible material and to stiffen the device. In some embodiments, the fifth layer of biocompatible material may later become the backing layer (e.g., backing layer 116) of the implantable piezoelectric ultrasound stimulator device.

In some embodiments, a thickness of the fifth layer of biocompatible material (e.g., backing layer 116) may be selected to give the device sufficient stiffness while maintaining overall flexibility. For example, in some embodiments, the implantable piezoelectric ultrasound stimulator device may require sufficient stiffness for ease of surgical implantation without the need for using an insertion shuttle. In some embodiments, the thickness of the fifth layer of biocompatible material (e.g., backing layer 116) may be approximately 15 μm, though the disclosure is not so limited.

In some embodiments, the structure of 1260 may be subjected to a post-exposure bake.

In 1263, the third substrate comprising the PET film and PVA layer may be removed. For example, the bonded substrates of the structure shown in 1260 may be soaked briefly (e.g., in 2-propanol) to dissolve the PVA sacrificial layer, thereby removing the PVA layer and the PET film.

In 1266, the second substrate may be removed from the structure of 1263. For example, the first layer of biocompatible material (e.g., SU-8) may be blanket dry-etched (e.g., in CF₄/O₂ for a minute) to expose the release layer (e.g., Omnicoat layer) and release the device from the second substrate. In some embodiments, once the first layer of biocompatible material is etched, the structure of 1263 may be soaked to dissolve the release layer, and then rinsed (e.g., in 2-propanol and DI water) until clean. In some embodiments, the resulting structure may then be arranged under a shadow mask and dry-etched to expose the metal bond pads, resulting in a finished implantable piezoelectric ultrasound stimulator device. The exposed metal bond pads may then be used to connect the implantable piezoelectric ultrasound stimulator device to other circuitry or instrumentation.

The above discussion provides examples of implantable piezoelectric ultrasound stimulator devices, associated structures and systems, and associated methods for making the example implantable piezoelectric ultrasound stimulator devices. As discussed above, the example implantable piezoelectric ultrasound stimulator devices may be micron-sized. As further discussed above, the example implantable piezoelectric ultrasound stimulator devices may be capable of modulating neuronal activity in deep subcortical regions, and of stimulating nigrostriatal dopamine production across long-range projections, as just some examples.

The discussion above demonstrates the scalable microfabrication of example implantable piezoelectric ultrasound stimulator devices, including using biocompatible materials, such as using KNN for the piezoelectric film and using SU-8 to encapsulate the elements of the device.

The discussion above further demonstrates that the resonance frequency of an example implantable piezoelectric ultrasound stimulator device may be controlled within a range of 0.2-1 MHz by manipulating the size of the cavity (e.g., cavity 112).

The discussion above demonstrates that the example implantable piezoelectric ultrasound stimulator devices discussed herein have consistent resonant behavior and minimal heating while placed in acoustic and thermal mediums mimicking the brain.

The example implantable piezoelectric ultrasound stimulator devices disclosed herein are also low power. For example, implantable piezoelectric ultrasound stimulator device 215, when operating at its resonant frequency, drew 0.2 mA of current with 10V peak-to-peak of applied voltage.

At a power consumption of 0.2 mW, implantable piezoelectric ultrasound stimulator device 215 generated ultrasound pressures of 100 kPa at resonance frequency in pulsed and continuous waves within its stimulation region, and evoked modulation of cell activity without causing thermogenic effects on nearby cells. The modulation of brain circuitry with a pressure of 100 kPa around 500 kHz is consistent with literature of in vivo experiments with mice using similar ultrasound driving parameters. Example implantable piezoelectric ultrasound stimulator device 215 elicited neuronal excitation in the hippocampus ex vivo, induced activity-dependent gene expression in hippocampal cells of an anesthetized mouse, and modulated dopaminergic neurons in the SNc to elicit precise timing of striatal dopamine release. All of this presents the example implantable piezoelectric ultrasound stimulator devices disclosed herein as potent neuromodulatory tools.

The example implantable piezoelectric ultrasound stimulator devices disclosed herein are also highly customizable. For example, as previously discussed, amplitude and frequency of generated ultrasound waves may be customized by modifying the diameter of the cavity (e.g., cavity 112) of the device when making the device, by modifying the voltage applied to the device in operation, by modifying the voltage signal applied to the device (e.g., sinusoidal vs. pulsed) in operation, and/or by modifying the frequency of the voltage signal applied to the device in operation.

The length of the implantable piezoelectric ultrasound stimulator device may also be modified depending on the region of the brain and or the type of living body into which the implantable piezoelectric ultrasound stimulator device is to be implanted. For example, the length of the implantable piezoelectric ultrasound stimulator device may be made to be short when the device is to be implanted into a brain region of a smaller animal, such as a mouse, or into an outer surface region of human brain. By contrast, the length of the implantable piezoelectric ultrasound stimulator device may be made to be long when the device is to be implanted into a deeper subcortical region of the human brain.

As previously discussed, the example implantable piezoelectric ultrasound stimulator devices disclosed herein may be utilized for chronic implantation. For example, as discussed with respect to FIGS. 7A-8C, aging and/or fatigue of the device may be compensated for by adjusting the voltage applied to the device. Based on studies of the durability of the device, including those discussed with respect to FIGS. 7A-8C, the device is expected to be capable of surviving the duration of a month-long chronic implantation.

Although a single ultrasound element (single pMUT) was discussed above with respect to FIGS. 1A-12, the disclosure is not so limited. Process 1200 of FIGS. 12A and 12B may be utilized to make an implantable piezoelectric ultrasound stimulator device with any number of electrodes, cavities, and piezoelectric films.

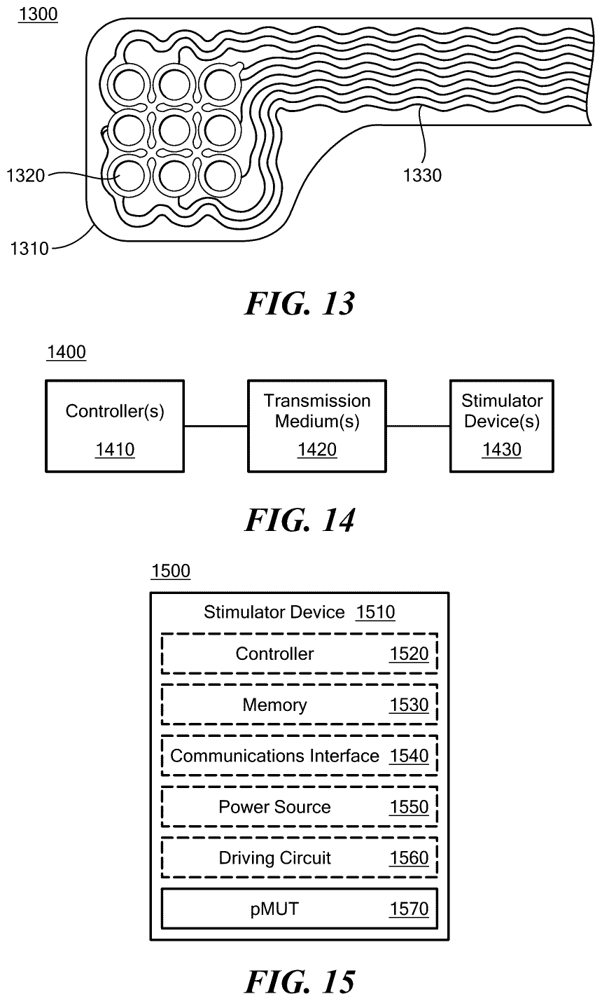

For example, FIG. 13 is an illustration 1300 of an example implantable piezoelectric ultrasound stimulator device 1310 comprising an array of nine piezoelectric elements 1320. Although nine piezoelectric elements are shown in FIG. 13, any number of piezoelectric elements may be provided in an implantable piezoelectric ultrasound stimulator device. Inclusion of multiple piezoelectric elements may be useful in applications where a higher amplitude of ultrasound waves is required to be generated. In some embodiments, all the multiple piezoelectric elements may be driven together with the same power source.

In some embodiments, some (or all) of the multiple piezoelectric elements may be independently controlled by a controller. For example, separate conductive traces 1330 may be coupled to different ones of the multiple piezoelectric elements, such that some (or all) of the multiple piezoelectric elements may be independently controlled. Allowing independent control of multiple piezoelectric elements may allow the different piezoelectric elements to be driven at different voltages and/or frequencies. For example, in some embodiments, different piezoelectric elements may be driven differently so as to create constructive and/or destructive interference between ultrasound waves. This may allow directional beams to be formed, which may provide further control over the amplitude, frequency, and/or direction at which to stimulate neurons in locations near the implantable piezoelectric ultrasound stimulator device.

FIG. 14 is a diagram of an example system 1400 for controlling an implantable piezoelectric ultrasound stimulator device. As shown in FIG. 14, one or more implantable piezoelectric ultrasound stimulator devices 1430 may be controlled by one or more controllers 1410 over one or more transmission mediums 1420. Implantable piezoelectric ultrasound stimulator device(s) 1430 may comprise one or more of any of the example implantable piezoelectric ultrasound stimulator devices disclosed herein, for example.

In some embodiments, implantable piezoelectric ultrasound stimulator device(s) 1430 may be controlled by one or more controllers 1410 over one or more transmission mediums 1420. For example, in some embodiments, an implantable piezoelectric ultrasound stimulator device, such as implantable piezoelectric ultrasound stimulator device 215 of FIGS. 2A-2C, may be controlled by one or more controllers (e.g., PCB 225) over one or more transmission mediums (e.g., ribbon cable 220).

Although FIG. 2A shows a PCB 225 as the controller, and a ribbon cable 220 as the transmission medium, the disclosure is not so limited. In some embodiments, a controller 1410 may comprise power supply circuitry for supplying power to the implantable piezoelectric ultrasound stimulator device(s). For example, in some embodiments, a controller 1410 may comprise circuitry capable of generating and delivering a certain type of voltage wave (e.g., a voltage wave of a certain amplitude, certain frequency, certain waveform). In some embodiments, a controller 1410 may comprise circuitry capable of generating and delivering one of a variety of different types of voltage waves, such as voltage waves having certain forms (e.g., sinusoidal, pulsed), frequencies, and/or amplitudes, for example.

In some embodiments, a controller 1410 may include a digital controller or processor. In some embodiments, such a digital controller or processor may be configured to execute instructions. Processors or controllers suitable for the execution of instructions may include, by way of example, both general and special purpose (e.g., application specific integrated circuit (ASIC)) processors or controllers. In some embodiments, a controller 1410 may include one or more memories, such as NOR and/or NAND flash memory devices, read only memory (ROM) devices, random access memory (RAM) devices, etc. In some embodiments, the one or more memories may store instructions that can be executed by a processor or digital controller of controller 1410 to provide a particular voltage waveform to the implantable piezoelectric ultrasound stimulator device(s) over transmission medium(s) 1420. In some embodiments, a controller 1410 may include input and/or output connections that allow a user or other system to program the device. For example, a user may select a particular voltage waveform for a controller 1410 to provide to the implantable piezoelectric ultrasound stimulator device(s).

Although FIGS. 2A and 2B show a ribbon cable 220 as the transmission medium for transmitting voltages to an implantable piezoelectric ultrasound stimulator device 215, the disclosure is not so limited. Any type of cable with conductive traces suitable for transmitting voltages to the electrodes of the implantable piezoelectric ultrasound stimulator devices may be used.

In some embodiments, a stimulator device 1430 may also comprise circuitry, such as a digital controller or processor, one or more memories, power driving circuitry, one or more communications interfaces, and/or one or more power supplies (see, e.g., FIG. 15), that may also be implantable in a living body. For example, these components may be further miniaturized and encapsulated in a biocompatible polymer along with the pMUT elements of the implantable piezoelectric ultrasound stimulator device, such that they may all be implanted into a living body. In such an embodiment, a remote control device may communicate wirelessly with a wireless communications interface in the implantable piezoelectric ultrasound stimulator device, so as to program and/or control settings and ultrasound wave generation of the implantable piezoelectric ultrasound stimulator device. Alternatively, in some embodiments, certain components (e.g., processor, memory, driving circuitry) may be implantable with the pMUT elements, and controllable by an external control device over a hard-wired communications medium.

FIG. 15 is a diagram 1500 of an example implantable piezoelectric stimulator device 1510. In some embodiments, an implantable piezoelectric stimulator device 1510 may comprise only a piezoelectric element (e.g., pMUT) and conductive traces within the biocompatible polymer, as previously discussed with respect to FIG. 2A. In some embodiments, voltages may be supplied to electrodes of the pMUT by an external controller over a transmission medium (e.g., ribbon cable 220).

In some embodiments, an implantable piezoelectric stimulator device 1510 may also include one or more controllers 1520, one or more memories 1530, one or more communications interfaces 1540, one or more power sources 1550, and/or one or more driving circuits 1560. For example, in some embodiments an implantable piezoelectric stimulator device 1510 may include one or more memories 1530 that are programmable by an external control device over one or more communications interfaces 1540. The one or more memories may include, for example, NOR and/or NAND flash memory devices, EPROM or EEPROM memory devices, ROM memory devices, RAM memory devices, or the like.

In some embodiments, one or more memories 1530 may be programmed and may include instructions that may get executed by one or more controllers 1520 included in implantable piezoelectric ultrasound stimulator device 1510. A controller 1520 may include, for example, one or more general or special purpose (e.g., application specific integrated circuit (ASIC)) processors or controllers.

In some embodiments, an implantable piezoelectric ultrasound stimulator device 1510 may include one or more power sources 1550. Power source(s) 1550 may include, for example, one or more batteries, such as one or more lithium polymer batteries.

In some embodiments, an implantable piezoelectric ultrasound stimulator device 1510 may include one or more driving circuits 1560 for driving one or more ultrasound elements (e.g., pMUTs 1570) of the implantable piezoelectric ultrasound stimulator device. In some embodiments, driving circuit(s) 1560 may be controlled by controller(s) 1520 to provide a desired voltage signal (e.g., voltage of a desired amplitude, frequency, and/or waveform shape) to one or more ultrasound elements (e.g., pMUTs) of the implantable piezoelectric ultrasound stimulator device. For example, controller(s) 1520 may be programmed to automatically adjust a voltage level applied to the one or ore more ultrasound elements based on an amount of time the device has been implanted or operated, so as to compensate for device fatigue and/or aging.

In some embodiments, an implantable piezoelectric ultrasound stimulator device 1510 may be programmed with certain settings before being implanted into a living body, such that no further external control of the implantable piezoelectric ultrasound stimulator device is provided once the device is implanted.

In some embodiments, an implantable piezoelectric ultrasound stimulator device may include one or more communications interfaces 1540 for communicating with an external control device. In some embodiments, communication interface(s) 1540 may communicate with the external control device over a hard-wired transmission medium, such as a universal serial bus (USB) cable, an Inter-Integrated Circuit (I2C) interface, or the like. In some embodiments, communication interface(s) 1540 may communicate with the external control device over a wireless transmission medium, such as through a WiFi interface, a Bluetooth interface, a cellular (e.g., 5G) interface, or the like.

As discussed herein, implantable ultrasound neurostimulation using the example implantable piezoelectric ultrasound stimulator devices disclosed herein may offer several advantages compared to other neurostimulation modalities. For example, unlike electrode-based deep brain stimulation, the example implantable piezoelectric ultrasound stimulator devices disclosed herein have no exposed electrochemical area, thus avoiding biofouling and corrosion. Moreover, compared to the cross-sectional footprint (in the mm range) and rigid form factor of devices used in DBS, the implantable piezoelectric ultrasound stimulator devices disclosed herein may be much smaller, yet still capable of precise and potent stimulation. Furthermore, as glial scar tissue forms in response to the implant, the acoustic properties of the encapsulating tissue change negligibly, and therefore retain desirable properties as the propagating medium for the effective delivery of ultrasound energy. Moreover, because the example implantable piezoelectric ultrasound stimulator devices disclosed herein are implanted adjacent to target neurons, the ultrasound generated may bypass the skull and may be focused to a volume that may be less than 100 μm³, thereby avoiding off-target activation from scattering and reflections. Using the example implantable piezoelectric ultrasound stimulator devices disclosed herein to generate ultrasounds for neurostimulation also offers the prospect of non-genetic and cell-specific modulation through its large stimulation parameter space, which has been demonstrated by selecting different pulse repetition frequencies (PRF).

As used herein, the term “processor” or “controller” is used to describe electronic circuitry that performs a function, an operation, or a sequence of operations. The function, operation, or sequence of operations can be hard coded into the electronic circuit or soft coded by way of instructions held in a memory device. The function, operation, or sequence of operations can be performed using digital values or using analog signals. In some embodiments, the processor or controller can be embodied in an application specific integrated circuit (ASIC), which can be an analog ASIC or a digital ASIC, in a microprocessor with associated program memory and/or in a discrete electronic circuit, which can be analog or digital. A processor or controller can contain internal processors or modules that perform portions of the function, operation, or sequence of operations. Similarly, a module can contain internal processors or internal modules that perform portions of the function, operation, or sequence of operations of the module.

While electronic circuits shown in figures herein may be shown in the form of analog blocks or digital blocks, it will be understood that the analog blocks can be replaced by digital blocks that perform the same or similar functions and the digital blocks can be replaced by analog blocks that perform the same or similar functions. Analog-to-digital or digital-to-analog conversions may not be explicitly shown in the figures but should be understood.

Various embodiments of the devices, structures, systems, and methods are described herein with reference to the related drawings. Alternative embodiments can be devised without departing from the scope of the described concepts. It is noted that various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. As an example of an indirect positional relationship, references in the present description to element or structure A over element or structure B include situations in which one or more intermediate elements or structures (e.g., element C) is between elements A and B regardless of whether the characteristics and functionalities of elements A and/or B are substantially changed by the intermediate element(s).

Furthermore, it should be appreciated that relative, directional or reference terms (e.g. such as “above,” “below,” “left,” “right,” “top,” “bottom,” “vertical,” “horizontal,” “front,” “back,” “rearward,” “forward,” etc.) and derivatives thereof are used only to promote clarity in the description of the figures. Such terms are not intended as, and should not be construed as, limiting. Such terms may simply be used to facilitate discussion of the drawings and may be used, where applicable, to promote clarity of description when dealing with relative relationships, particularly with respect to the illustrated embodiments. Such terms are not, however, intended to imply absolute relationships, positions, and/or orientations. For example, with respect to an object or structure, an “upper” or “top” surface can become a “lower” or “bottom” surface simply by turning the object over. Nevertheless, it is still the same surface and the object remains the same. Also, as used herein, “and/or” means “and” or “or,” as well as “and” and “or.”

The terms “disposed over,” “overlying,” “atop,” “on top,” “positioned on” or “positioned atop” mean that a first element, such as a first structure, is present on a second element, such as a second structure, where intervening elements or structures (such as an interface structure) may or may not be present between the first element and the second element. The term “direct contact” means that a first element, such as a first structure, and a second element, such as a second structure, are connected without any intermediary elements or structures between the interface of the two elements. The term “connection” can include an indirect connection and a direct connection.

In the foregoing detailed description, various features are grouped together in one or more individual embodiments for the purpose of streamlining the disclosure. This method of disclosure is not to be interpreted as reflecting an intention that each claim requires more features than are expressly recited therein. Rather, inventive aspects may lie in less than all features of each disclosed embodiment.

References in the disclosure to “one embodiment,” “an embodiment,” “some embodiments,” or variants of such phrases indicate that the embodiment(s) described can include a particular feature, structure, or characteristic, but every embodiment can include the particular feature, structure, or characteristic. Moreover, such phrases are not necessarily referring to the same embodiment(s). Further, when a particular feature, structure, or characteristic is described with reference to one embodiment, knowledge of one skilled in the art may be relied upon to affect such feature, structure, or characteristic in connection with other embodiments whether or not explicitly described.

The terms “approximately,” “substantially,” or “about” may be used herein to mean within +/−30% of a target value in some embodiments, within +/−20% of a target value in some embodiments, within +/−10% of a target value in some embodiments, within +/−5% of a target value in some embodiments, and within +/−2% of a target value in some embodiments. The aforementioned terms may include the target value. The aforementioned terms may include an exact matching of values.

Moreover, it should be understood when discussing dimensional values herein that such values may be approximate. For example, variations from a listed value may occur due to, for example, manufacturing tolerances, variations due to ambient temperature changes, and the like. In some embodiments, these variations may include variations of up to +/−30% of a target value in some embodiments, up to +/−20% of a target value in some embodiments, up to +/−10% of a target value in some embodiments, up to +/−5% of a target value in some embodiments, and up to +/−2% of a target value in some embodiments.

The disclosed subject matter is not limited in its application to the details of construction and to the arrangements of the components set forth in the following description or illustrated in the drawings. The disclosed subject matter is capable of other embodiments and of being practiced and carried out in various ways. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

All publications and references cited herein are expressly incorporated herein by reference in their entirety.

1. An implantable piezoelectric ultrasound stimulator device, comprising:
a first electrode;
a second electrode;
a piezoelectric film disposed between the first electrode and the second electrode; and
a biocompatible polymer that encapsulates the first electrode, the second electrode, the piezoelectric film; and a cavity.

2. The implantable piezoelectric ultrasound stimulator device of claim 1, wherein the biocompatible polymer comprises SU-8.

3. The implantable piezoelectric ultrasound stimulator device of claim 1, wherein the biocompatible polymer comprises:
a backing layer;
a cavity layer forming the cavity;
a membrane layer; and
a top layer.

4. The implantable piezoelectric ultrasound stimulator device of claim 1, wherein the piezoelectric film comprises a biocompatible ceramic.

5. The implantable piezoelectric ultrasound stimulator device of claim 1, wherein the piezoelectric film comprises potassium sodium niobate (KNN).

6. The implantable piezoelectric ultrasound stimulator device of claim 3, wherein the implantable piezoelectric ultrasound stimulator device comprises a piezoelectric micromachined ultrasound transducer (pMUT), the pMUT configured to generate and direct ultrasound waves in a direction away from an exposed planar surface of the top layer.

7. The implantable piezoelectric ultrasound stimulator device of claim 1, wherein the implantable piezoelectric ultrasound stimulator device is configured to stimulate neurons in the brain.

8. The implantable piezoelectric ultrasound stimulator device of claim 1, wherein the implantable piezoelectric ultrasound stimulator device is less than 50 micrometers (μm) thick and less than 200 μm wide.

9. The implantable piezoelectric ultrasound stimulator device of claim 1, wherein the cavity is positioned on one side of the piezoelectric film and is filled with air.

10. The implantable piezoelectric ultrasound stimulator device of claim 1, wherein the first electrode, the second electrode, the cavity, and the piezoelectric film together form one ultrasound element of an array of ultrasound elements in the implantable piezoelectric ultrasound stimulator device, each of the ultrasound elements in the implantable piezoelectric ultrasound stimulator device comprising at least two electrodes, a cavity, and a piezoelectric film.

11. A system, comprising:
the implantable piezoelectric ultrasound stimulator device of claim 1;
a controller; and
a power source.

12. The system of claim 11, wherein the controller is configured to deliver a voltage from the power source to the first electrode or the second electrode.

13. The system of claim 12, wherein the voltage is one of a sinusoidal voltage or a pulsed voltage.

14. The system of claim 13, wherein the controller is further configured to control a frequency at which the sinusoidal voltage or pulsed voltage is delivered.

15. The system of claim 11,
wherein the first electrode, the second electrode, the cavity, and the piezoelectric film together form one ultrasound element of an array of ultrasound elements in the implantable piezoelectric ultrasound stimulator device, each of the ultrasound elements in the implantable piezoelectric ultrasound stimulator device comprising at least two electrodes, a cavity, and a piezoelectric film, and
wherein the controller is configured to individually control the ultrasound elements in the array to form an ultrasound beam focused in a specific direction.

16. The system of claim 11, wherein the controller and the power source are implantable, and the controller is configured to communicate wirelessly with a control device.

17. The system of claim 11, wherein the controller is configured to control the implantable piezoelectric ultrasound stimulator device over a cable.

18. A method of making an implantable piezoelectric ultrasound stimulator device, the method comprising:
providing a stack of layers on top of a first substrate, the stack of layers comprising at least a first electrode layer in contact with the top of the first substrate and comprising at least one first electrode, a second electrode layer comprising at least one second electrode, and a piezoelectric layer between the first electrode layer and the second electrode layer and comprising at least one piezoelectric film;
coating the stack of layers and a portion of the first substrate with an anchor material;
undercutting the stack of layers by removing at least a portion of the first substrate;
removing the stack of layers and at least a portion of the anchor material from the first substrate;
pressing the stack of layers and the at least a portion of the anchor material onto a first layer of a biocompatible polymer, the first layer of the biocompatible polymer positioned atop a release layer and a second substrate;
removing the at least a portion of the anchor material from the stack of layers;
coating a second layer of the biocompatible polymer onto the stack of layers and the first layer of the biocompatible polymer, leaving openings to the at least one first electrode and the at least one second electrode;
depositing and etching metal interconnects and bond pads for connecting the at least one first electrode and the at least one second electrode to external circuitry;
coating a third layer of the biocompatible polymer onto the metal interconnects and the second layer of the biocompatible polymer;
removing the second substrate from the release layer; and
removing the release layer.

19. The method of claim 18, further comprising:
patterning a cavity on top of the third layer of the biocompatible polymer;
coating a fourth layer of the biocompatible polymer onto the third layer of the biocompatible polymer, except where the cavity was patterned;
bonding the fourth layer of the biocompatible polymer to a fifth layer of the biocompatible polymer on a third substrate; and
removing the third substrate.

20. The method of claim 19, wherein the biocompatible polymer comprises SU-8 and the at least one piezoelectric film comprises potassium sodium niobate (KNN).
